SEQUENCE LISTING (24 sequences)

SEQ ID NO: 1 - DNA, 21 bp, mus musculus
gcagattggatcggaaagtaa

SEQ ID NO: 2 - DNA, 18 bp, mus musculus
gatgagaagaggattata

SEQ ID NO: 3 - DNA, 21 bp, Artificial Sequence
  misc_feature: control shRNA_1
cctaaggttaagtcgccctcg

SEQ ID NO: 4 - DNA, 21 bp, mus musculus
cgatcttcacactgactcttt

SEQ ID NO: 5 - DNA, 21 bp, mus musculus
taccactgtcgcaaccgaaat

SEQ ID NO: 6 - DNA, 18 bp, Artificial Sequence
  misc_feature: control shRNA_2
caagatgaagagcaccaa

SEQ ID NO: 7 - DNA, 21 bp, mus musculus
cgccttcacttgaccataatt

SEQ ID NO: 8 - DNA, 18 bp, mus musculus
gcacttgaagacttctaa

SEQ ID NO: 9 - DNA, 21 bp, mus musculus
tggcctgtggagtaaggtcaa

SEQ ID NO: 10 - DNA, 21 bp, mus musculus
gaagcagaggacaagttccca

SEQ ID NO: 11 - DNA, 20 bp, mus musculus
gaaacccccggattagagcc

SEQ ID NO: 12 - DNA, 21 bp, mus musculus
gagcaaaggtctccttgaggt

SEQ ID NO: 13 - DNA, 21 bp, mus musculus
gcacctgactgatgctgagaa

SEQ ID NO: 14 - DNA, 21 bp, mus musculus
ttcatcggcgttcacctttcc

SEQ ID NO: 15 - DNA, 22 bp, mus musculus
atgatgaacaccgaccttagca

SEQ ID NO: 16 - DNA, 20 bp, mus musculus
cggagggctctttggatttc

SEQ ID NO: 17 - DNA, 19 bp, homo sapiens
tggatgatctcaagggcac

SEQ ID NO: 18 - DNA, 20 bp, homo sapiens
tcagtggtatctggaggaca

SEQ ID NO: 19 - DNA, 20 bp, homo sapiens
gcaagaaggtgctgacttcc

SEQ ID NO: 20 - DNA, 20 bp, homo sapiens
accatcacgttacccaggag

SEQ ID NO: 21 - DNA, 21 bp, homo sapiens
ctgaggagaagtctgccgtta

SEQ ID NO: 22 - DNA, 20 bp, homo sapiens
agcatcaggagtggacagat

SEQ ID NO: 23 - DNA, 20 bp, homo sapiens
acccagaagactgtggatgg

SEQ ID NO: 24 - DNA, 20 bp, homo sapiens
ttcagctcagggatgacctt

(19) United States
(12) Patent Application Publication
(10) Pub. No.: US 2024/0100049 A1
(43) Pub. Date: Mar. 28, 2024

(54) REACTIVATION OF EMBRYONIC AND FETAL HEMOGLOBIN

(71) Applicant: The Henry M. Jackson Foundation for the Advancement of Military Medicine, Inc., Bethesda, MD (US)

(72) Inventor: Yang Du, Bethesda, MD (US)

(21) Appl. No.: 18/274,565

(22) PCT Filed: Jan. 27, 2022
(86) PCT No.: PCT/US2022/014109

§ 371 Date: Jul. 27, 2023

Related U.S. Application Data
(60) Provisional application No. 63/142,841, filed on Jan. 28, 2021.

(51) Int. Cl. A61K 31/506; A61K 31/519; A61P 7/06

(52) U.S. Cl. CPC A61K 31/506; A61K 31/519; A61P 7/06

(57) ABSTRACT

Provided herein are methods for treating β-globinopathies, such as sickle cell disease and β-thalassemia, by inducing fetal hemoglobin expression and/or embryonic hemoglobin expression.

### CROSS-REFERENCE TO RELATED APPLICATIONS

This application claims priority to U.S. Provisional Application No. 63/142,841, filed Jan. 28, 2021, which is incorporated herein by reference for all purposes.

### STATEMENT REGARDING FEDERALLY SPONSORED RESEARCH OR DEVELOPMENT

This invention was made with government support under RAMP310534 and R086414218 awarded by the Uniformed Services University of the Health Sciences. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Jan. 27, 2022, is named SequenceListing_ST25.txt and is 4,096 bytes in size.

### FIELD

Provided herein are methods for treating β-globinopathies, such as sickle cell disease and 3-thalassemia, by inducing fetal hemoglobin expression and/or embryonic hemoglobin expression.

### BACKGROUND

β-globinopathies, including sickle cell disease (SCD) and β-thalassemia, are among the most common monogenic disorders worldwide. SCD is due to the synthesis of abnormal hemoglobin, which tends to aggregate under low oxygen conditions, while reduced expression or absence of β-globins cause β-thalassemia. Large numbers of children are born with β-globinopathies each year with approximately 300,000 with SCD and 40,000 with β-thalassemia.

Treatment and management of SCD is mostly focused on pain relief and blood transfusions. In β-thalassemias, blood transfusions are often needed throughout the patient's life. However, repeated blood transfusions cause iron overload, which is a major complication affecting both cardiovascular and liver function. Therefore, other therapeutic options are being actively explored, including bone marrow transplantation and treatments to reactivate the expression of embryonic and fetal hemoglobins. However, both the high cost associated with bone marrow transplantation and the difficulty in finding matched donors has prevented the procedure's broad usage. Fetal hemoglobin expression can be induced by non-targeted drug treatments such as hydroxyurea. However, the induction efficiency by hydroxyurea is low, and the treatment is only effective for some patients.

Other approaches being sought aim at targeting the repressors of HbF expression. In adult erythroid progenitors, fetal and embryonic hemoglobin expression is thought to be maintained in a repressed state by co-repressor complexes containing BCL11A and LRF. Genetically targeting components of these complexes in erythroid cells in vitro or in mouse models has been shown to de-repress embryonic and fetal hemoglobin expression. However, efficient small molecule inhibitors of this complex with low toxicity remain to be identified.

Therefore, a need exists for therapeutic agents that can induce fetal and/or embryonic hemoglobin expression, thereby treating β-globinopathies (such as SCD and β-thalassemia).

### SUMMARY

Provided herein are methods of treating a β-globinopathy comprising administering to the subject a therapeutically effective amount of a compound or compounds that modulates activity of BMI1 protein.

Provided herein are methods of treating a β-globinopathy comprising administering to the subject a therapeutically effective amount of a compound or compounds that modulates the interaction between MEN1 and KMT2A.

### BRIEF DESCRIPTION OF THE DRAWINGS

Bmi1 knockdown increased embryonic globin expression levels in MEL cells. FIG. 1A shows real-time RT-PCR analysis of Hbb-y and Hbb-bh1 mRNA levels in MEL cells at 96 hours after infection with lentiviral shRNAs targeting Bmi1 (Bmi1-sh1 and -sh2) or non-targeting control lentiviral shRNA (NC-sh). Relative expression levels were calculated by normalizing to Rpl4 mRNA levels in the same sample and also to cells infected with NC-sh virus. FIG. 1B shows western blotting analysis of protein levels of BMI1 and control β-ACTIN in MEL cells at 72 hours after infection with the indicated lentiviral shRNAs. The data shows significantly reduced BMI1 protein levels in cells transduced by the two Bmi1-specific shRNAs.

FIG. 2 provides data demonstrating that PTC596 increased embryonic globin expression levels in MEL cells. Real-time RT-PCR analysis of Hbb-y, Hbb-bh1, and Hbb-b1/2 mRNA levels in MEL cells after 48 hours of treatment with PTC596 at indicated concentration or with vehicle DMSO are shown. Relative expression levels were calculated by normalizing to Rpl4 mRNA levels in the same sample and also to cells treated with DMSO.

Men1 knockdown increased embryonic globin expression levels in MEL cells. FIG. 3A shows real-time RT-PCR analysis of Hbb-y and Hbb-bh1 mRNA levels in MEL cells at 96 hours after infection with lentiviral shRNAs targeting Men1 (Men1-sh1 and -sh2) or non-targeting control lentiviral shRNA (NC-sh). Relative expression levels were calculated by normalizing to Rpl4 mRNA levels in the same sample and also to cells infected with NC-sh virus. FIG. 3B shows the western blotting analysis of protein levels of MEN1 and control β-ACTIN in MEL cells at 72 hours after infection with the indicated lentiviral shRNAs. The data shows significantly reduced MEN1 protein levels in cells transduced by the two Men1-specific shRNAs.

Kmt2a knockdown increased embryonic globin expression levels in MEL cells. FIG. 4A shows real-time RT-PCR analysis of Hbb-y and Hbb-bh1 mRNA levels in MEL cells at 96 hours after infection with lentiviral shRNAs targeting Kmt2a (Kmt2a-sh1 and -sh2) or non-targeting control lentiviral shRNA (NC-sh). Relative expression levels were calculated by normalizing to Rpl4 mRNA levels in the same sample and also to cells infected with NC-sh virus. FIG. 4B shows the western blotting analysis of protein levels of KMT2A and control β-ACTIN in MEL cells at 72 hours after infection with the indicated lentiviral shRNAs. The data shows significantly reduced KMT2A protein levels in cells transduced by the two Kmt2a-specific shRNAs.

FIG. 5 provides data demonstrating that MENIN inhibitor MI-3454 increased embryonic globin expression levels in MEL cells. Real-time RT-PCR analysis of Hbb-y, Hbb-bh1, and Hbb-b1/2 mRNA levels in MEL cells after 48 hours of treatment with MI-3454 (MI) at indicated concentration or with vehicle DMSO are shown. Relative expression levels were calculated by normalizing to Rpl4 mRNA levels in the same sample and also to cells treated with DMSO.

FIG. 6 provides data demonstrating that MENIN inhibitor SNDX-5613 increased embryonicglobin expression levels in MEL cells. Real-time RT-PCR analysis of Hbb-bh1 mRNA levels in MEL cells after 48 hours of treatment with SNDX-5613 (Sn) at indicated concentration or with Vehicle DMSO. Relative expression levels were calculated by normalizing to Rpl4 mRNA levels in the same sample and also to cells treated with DMSO.

FIG. 7 is a cytospin analysis showing erythroblasts and reticulocytes in cultures of human CD34⁺ HSPCs treated with PTC596 and MI-3454 and induced to undergo erythroid differentiation for 14 days. Cells were stained using Hema 3 staining system (Fisher Scientific). Scale bar, 5 μM.

FIG. 8 provides data demonstrating that PTC596 and MI-3454 significantly increased fetal and embryonic globin expression levels in human erythroid cells. Human CD34⁺ HSPCs treated with PTC596 (PTC) or MI-3454 (MI) at indicated concentrations or vehicle (DMSO) were induced to undergo erythroid differentiation. Cells were harvested after 14 days of differentiation for real-time RT-PCR analysis of human fetal γ (FIG. 8A), embryonic a (FIG. 8B), and adult β (FIG. 8C) globin mRNA levels. Relative expression levels were calculated by normalizing to GAPDH mRNA levels in the same sample and also to cells treated with DMSO. FIG. 8D shows representative bar graphs showing γ-globin expression levels as a percentage of total β-like globin (β+γ+ε) expression levels after treatment with PTC596 and MI-3454 versus DMSO. *, p<0.05; **, p<0.01; ***, p<0.001; ****, p<0.0001 (two-tailed Student's t test).

FIG. 9 provides data demonstrating that PTC596 and MI-3454 significantly increased 7-Globin protein levels in human erythroid cells. Western blotting analysis of protein levels of 7-Globin, BMI1, MEN1, and control ACTIN in CD34⁺ HSPCs were treated with PTC596 (PTC) at indicated concentrations (FIG. 9A), MI-3454 (MI) at indicated concentrations (FIG. 9B), or DMSO and induced to undergo erythroid differentiation for 14 days.

FIG. 10 shows flow cytometry analysis of F-cells in cultures of human CD34⁺ HSPCs treated with PTC596 (PCT) at indicated concentrations and MI-3454 (MI) at indicated concentrations and induced to undergo erythroid differentiation for 14 days. Cells were fixed with glutaraldehyde, permeabilized, and stained with a PE-conjugated anti-HbF antibody. The percentages of F-cells are indicated.

### DETAILED DESCRIPTION

### Definitions

Unless defined otherwise, all technical and scientific terms used herein have the same meaning as is commonly understood by one of ordinary skill in the art. As used herein, the below terms have the following meanings unless specified otherwise. Any methods, devices and materials similar or equivalent to those described herein can be used in the practice of the compositions and methods described herein. The following definitions are provided to facilitate understanding of certain terms used frequently herein and are not meant to limit the scope of the present disclosure. All references referred to herein are incorporated by reference in their entirety.

It is noted here that as used in this specification and the appended claims, the singular forms “a,” “an,” and “the,” and the like include plural referents unless the context clearly dictates otherwise.

The term “about” or “approximately” means within ±30%, 20%, 15%, 10%, 9%, 8%, 7%, 6%, 5%, 4%, 3%, 2%, 1%, 0.5%, or 0.05% of a given value or range. In some embodiments, “about” means±5% of a given value or range. In some embodiments, “about” means±4% of a given value or range. In some embodiments, “about” means±3% of a given value or range. In some embodiments, “about” means±2% of a given value or range. In some embodiments, “about” means±1% of a given value or range. In another embodiment, about means±0.5% of a given value or range. In some embodiments, “about” means±0.05% of a given value or range.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line or a dashed line drawn through or perpendicular across the end of a line in a structure indicates a specified point of attachment of a group. Unless chemically or structurally required, no directionality or stereochemistry is indicated or implied by the order in which a chemical group is written or named.

The prefix “Cᵤ₋ᵥ” indicates that the following group has from u to v carbon atoms. For example, “C₁₋₆ alkyl” indicates that the alkyl group has from 1 to 6 carbon atoms. In another example, “C₁₋₄ alkyl” indicates that the alkyl group has from 1 to 4 carbon atoms.

“Alkyl” refers to an unbranched or branched saturated hydrocarbon chain. As used herein, alkyl has 1 to 20 carbon atoms (i.e., C₁₋₂₀ alkyl), 1 to 12 carbon atoms (i.e., C₁₋₁₂ alkyl), 1 to 8 carbon atoms (i.e., C₁₋₈ alkyl), 1 to 6 carbon atoms (i.e., C₁₋₆ alkyl) or 1 to 4 carbon atoms (i.e., C₁₋₄ alkyl). Examples of alkyl groups include, e.g., methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂) and tert-butyl (i.e., —C(CH₃)₃); and “propyl” includes n-propyl (i.e., —(CH₂)₂CH₃) and isopropyl (i.e., —CH(CH₃)₂).

Certain commonly used alternative chemical names may be used. For example, a divalent group such as a divalent “alkyl” group, a divalent “aryl” group, etc., may also be referred to as an “alkylene” group or an “alkylenyl” group, an “arylene” group or an “arylenyl” group, respectively. Also, unless indicated explicitly otherwise, where combinations of groups are referred to herein as one moiety, e.g., arylalkyl or aralkyl, the last-mentioned group contains the atom by which the moiety is attached to the rest of the molecule.

“Alkenyl” refers to an alkyl group containing at least one carbon-carbon double bond and having from 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl) or 2 to 4 carbon atoms (i.e., C₂₋₄ alkenyl). Examples of alkenyl groups include, e.g., ethenyl, propenyl, butadienyl (including 1,2-butadienyl and 1,3-butadienyl).

“Alkynyl” refers to an alkyl group containing at least one carbon-carbon triple bond and having from 2 to 20 carbon atoms (i.e., C₂₋₂₀ alkynyl), 2 to 8 carbon atoms (i.e., C₂₋₈ alkynyl), 2 to 6 carbon atoms (i.e., C₂₋₆ alkynyl) or 2 to 4 carbon atoms (i.e., C₂₋₄ alkynyl). The term “alkynyl” also includes those groups having one triple bond and one double bond.

“Alkoxy” refers to the group “alkyl-O—”. Examples of alkoxy groups include, e.g., methoxy, ethoxy, n-propoxy, iso-propoxy, n-butoxy, tert-butoxy, sec-butoxy, n-pentoxy, n-hexoxy and 1,2-dimethylbutoxy.

“Alkylthio” refers to the group “alkyl-S—”. “Alkylsulfinyl” refers to the group “alkyl-S(O)—”. “Alkylsulfonyl” refers to the group “alkyl-S(O)₂—”.

In some embodiments, “alkylcarbonyl” refers to —C(═O)-alkyl. In some embodiments, “alkoxycarbonyl” refers to —C(═O)(O)-alkyl. In some embodiments, “aminocarbonyl” refers to —C(═O)NH₂.

“Acyl” refers to a group —C(O)R^{y}, wherein R^{y} is hydrogen, alkyl, alkenyl, alkynyl, cycloalkyl, heterocyclyl, aryl, heteroalkyl or heteroaryl; each of which may be optionally substituted, as defined herein. Examples of acyl include, e.g., formyl, acetyl, cyclohexylcarbonyl, cyclohexylmethyl-carbonyl and benzoyl.

“Amido” refers to both a “C-amido” group which refers to the group —C(O)NR^{y}R^{z} and an “N-amido” group which refers to the group —NR^{y}C(O)R^{z}, wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein, or R^{y} and R^{z} are taken together to form a cycloalkyl or heterocyclyl; each of which may be optionally substituted, as defined herein.

“Amino” refers to the group —NR^{y}R^{z} wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein. In some embodiments, amino is —NH₂. In some embodiments, “alkylamino” refers to —NH(alkyl), wherein alkyl is as defined herein. In some embodiments, “dialkylamino” refers to —N(alkyl)₂, wherein alkyl is as defined herein.

“Amidino” refers to —C(NR^{y})(NR^{z}₂), wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include, e.g., phenyl, naphthyl, fluorenyl and anthryl. Aryl, however, does not encompass or overlap in any way with heteroaryl defined below. If one or more aryl groups are fused with a heteroaryl, the resulting ring system is heteroaryl regardless of the point of attachment. If one or more aryl groups are fused with a heterocyclyl, the resulting ring system is heterocyclyl regardless of the point of attachment.

“Arylalkyl” or “Aralkyl” refers to the group “aryl-alkyl-”.

In some embodiments, “B(OR₈)₂” refers to a radical of the formula: —B[(—OH)(—OH)] when R₈ is hydrogen; or, —B[(—OH)(—O—C₁₋₈alkyl)] when R₈ is independently hydrogen or C₁₋₈alkyl; or, —B[(—O—C₁₋₈alkyl)(—O—C₁₋₈alkyl)] when R₈ is C₁₋₈alkyl.

“Carbamoyl” refers to both an “O-carbamoyl” group which refers to the group —O—C(O)NR^{y}R^{z} and an “N-carbamoyl” group which refers to the group —NR^{y}C(O)OR^{z}, wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein.

“Carboxyl ester” or “ester” refer to both —OC(O)R^{x} and —C(O)OR^{x}, wherein R^{x} is alkyl, alkenyl, alkynyl, cycloalkyl, heterocyclyl, aryl, heteroalkyl or heteroaryl; each of which may be optionally substituted, as defined herein. In some embodiments, “carboxy” or “carboxyl” refers to —CO₂H.

“Cycloalkyl” refers to a saturated or partially unsaturated cyclic alkyl group having a single ring or multiple rings including fused, bridged and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Monocyclic groups include, for example, cyclopropyl, cyclobutyl, cyclopentyl, cyclohexyl, cycloheptyl and cyclooctyl. Polycyclic groups include, for example, bicyclo[2.2.1]heptanyl, bicyclo[2.2.2]octanyl, adamantyl, norbornyl, decalinyl, 7,7-dimethyl-bicyclo[2.2.1]heptanyl and the like. Further, the term cycloalkyl is intended to encompass any non-aromatic ring which may be fused to an aryl ring, regardless of the attachment to the remainder of the molecule. Still further, cycloalkyl also includes “spirocycloalkyl” when there are two positions for substitution on the same carbon atom, for example spiro[2.5]octanyl, spiro[4.5]decanyl, or spiro[5.5]undecanyl.

In some embodiments, “carbocycle” refers to a saturated, unsaturated, or aromatic ring in which each atom of the ring is a carbon atom. Carbocycle may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In some embodiments, the carbocycle is an aryl. In some embodiments, the carbocycle is a cycloalkyl. In some embodiments, the carbocycle is a cycloalkenyl. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl.

“Cycloalkylalkyl” refers to the group “cycloalkyl-alkyl-”.

“Cyanoalkyl” refers to, in some embodiments -alkyl-CN.

“Guanidino” refers to —NR^{y}C(═NR^{z})(NR^{y}R^{z}), wherein each R^{y} and R^{z} are independently hydrogen, alkyl, alkenyl, alkynyl, cycloalkyl, heterocyclyl, aryl, heteroalkyl or heteroaryl; each of which may be optionally substituted, as defined herein.

“Imino” refers to a group —C(NR^{y})R^{z}, wherein R^{y} and R^{z} are each independently hydrogen, alkyl, alkenyl, alkynyl, cycloalkyl, heterocyclyl, aryl, heteroalkyl or heteroaryl; each of which may be optionally substituted, as defined herein.

“Imido” refers to a group —C(O)NR^{y}C(O)R^{z}, wherein R^{y} and R^{z} are each independently hydrogen, alkyl, alkenyl, alkynyl, cycloalkyl, heterocyclyl, aryl, heteroalkyl or heteroaryl; each of which may be optionally substituted, as defined herein.

“Halogen” or “halo” refers to atoms occupying group VIIA of the periodic table, such as fluoro, chloro, bromo or iodo.

“Haloalkyl” refers to an unbranched or branched alkyl group as defined above, wherein one or more (e.g., 1 to 6, or 1 to 3) hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl and the like.

“Haloalkoxy” refers to an alkoxy group as defined above, wherein one or more (e.g., 1 to 6, or 1 to 3) hydrogen atoms are replaced by a halogen.

“Hydroxyalkyl” refers to an alkyl group as defined above, wherein one or more (e.g., 1 to 6, or 1 to 3) hydrogen atoms are replaced by a hydroxy group. A “mono-hydroxy-(C₁₋₄ alkyl)” refers to a C₁₋₄ alkyl group as defined above, wherein one hydrogen atom is replaced by a hydroxy group. A “di-hydroxy-(C₁₋₄ alkyl)” refers to a C₁₋₄ alkyl group as defined above, wherein two hydrogen atoms are replaced by hydroxy groups.

“Heteroalkyl” refers to an alkyl group in which one or more of the carbon atoms (and any associated hydrogen atoms) are each independently replaced with the same or different heteroatomic group, provided the point of attachment to the remainder of the molecule is through a carbon atom. The term “heteroalkyl” includes unbranched or branched saturated chain having carbon and heteroatoms. By way of example, 1, 2 or 3 carbon atoms may be independently replaced with the same or different heteroatomic group. Heteroatomic groups include, but are not limited to, —NR^{y}—, —O—, —S—, —S(O)—, —S(O)₂—, and the like, wherein R is hydrogen, alkyl, alkenyl, alkynyl, cycloalkyl, heterocyclyl, aryl, heteroalkyl or heteroaryl; each of which may be optionally substituted, as defined herein. Examples of heteroalkyl groups include, e.g., ethers (e.g., —CH₂OCH₃, —CH(CH₃)OCH₃, —CH₂CH₂OCH₃, —CH₂CH₂OCH₂CH₂OCH₃, etc.), thioethers (e.g., —CH₂SCH₃, —CH(CH₃)SCH₃, —CH₂CH₂SCH₃, —CH₂CH₂SCH₂CH₂SCH₃, etc.), sulfones (e.g., —CH₂S(O)₂CH₃, —CH(CH₃)S(O)₂CH₃, —CH₂CH₂S(O)₂CH₃, —CH₂CH₂S(O)₂CH₂CH₂OCH₃, etc.) and amines (e.g., —CH₂NR^{y}CH₃, —CH(CH₃)NR^{y}CH₃, —CH₂CH₂NR^{y}CH₃, —CH₂CH₂NR^{y}CH₂CH₂NR^{y}CH₃, etc., where R^{y} is hydrogen, alkyl, alkenyl, alkynyl, cycloalkyl, heterocyclyl, aryl, heteroalkyl, or heteroaryl; each of which may be optionally substituted, as defined herein). As used herein, heteroalkyl includes 1 to 10 carbon atoms, 1 to 8 carbon atoms, or 1 to 4 carbon atoms; and 1 to 3 heteroatoms, 1 to 2 heteroatoms, or 1 heteroatom.

“Heteroaryl” refers to an aromatic group having a single ring, multiple rings or multiple fused rings, with one or more ring heteroatoms independently selected from nitrogen, oxygen, and sulfur. As used herein, heteroaryl includes 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C_{3M} heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen and sulfur. In certain instances, heteroaryl includes 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heteroarylalkyl” refers to the group “heteroaryl-alkyl-”.

“Heterocyclyl” refers to a saturated or partially unsaturated cyclic alkyl group, with one or more ring heteroatoms independently selected from nitrogen, oxygen and sulfur. The term “heterocyclyl” includes heterocycloalkenyl groups (i.e., the heterocyclyl group having at least one double bond), bridged-heterocyclyl groups, fused-heterocyclyl groups and spiro-heterocyclyl groups. A heterocyclyl may be a single ring or multiple rings wherein the multiple rings may be fused, bridged or spiro, and may comprise one or more (e.g., 1 to 3) oxo (═O) or N-oxide (—O—) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 2 to 20 ring carbon atoms (i.e., C₂₋₂₀ heterocyclyl), 2 to 12 ring carbon atoms (i.e., C₂₋₁₂ heterocyclyl), 2 to 10 ring carbon atoms (i.e., C₂₋₁₀ heterocyclyl), 2 to 8 ring carbon atoms (i.e., C₂₋₈ heterocyclyl), 3 to 12 ring carbon atoms (i.e., C₃₋₁₂ heterocyclyl), 3 to 8 ring carbon atoms (i.e., C₃₋₈ heterocyclyl), or 3 to 6 ring carbon atoms (i.e., C₃₋₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiophenyl (i.e., thienyl), tetrahydropyranyl, thiomorpholinyl, thiamorpholinyl, 1-oxo-thiomorpholinyl and 1,1-dioxo-thiomorpholinyl. The term “heterocyclyl” also includes “spiroheterocyclyl” when there are two positions for substitution on the same carbon atom. Examples of the spiro-heterocyclyl rings include, e.g., bicyclic and tricyclic ring systems, such as 2-oxa-7-azaspiro[3.5]nonanyl, 2-oxa-6-azaspiro[3.4]octanyl and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl and isoindolinyl, where the heterocyclyl can be bound via either ring of the fused system.

In some embodiments, “heterocycle” refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, and 6- to 12-membered bridged rings. Each ring of a bicyclic heterocycle may be selected from saturated, unsaturated, and aromatic rings. The heterocycle may be attached to the rest of the molecule through any atom of the heterocycle, valence permitting, such as a carbon or nitrogen atom of the heterocycle. In some embodiments, the heterocycle is a heteroaryl. In some embodiments, the heterocycle is a heterocycloalkyl. In an exemplary embodiment a heterocycle, e.g., pyridyl, may be fused to a saturated or unsaturated ring, e.g., cyclohexane, cyclopentane, or cyclohexene.

“Heterocyclylalkyl” refers to the group “heterocyclyl-alkyl-.”

“Oxime” refers to the group —CR^{y}(═NOH) wherein R^{y} is hydrogen, alkyl, alkenyl, alkynyl, cycloalkyl, heterocyclyl, aryl, heteroalkyl or heteroaryl; each of which may be optionally substituted, as defined herein.

“Sulfonyl” refers to the group —S(O)₂R^{y}, where R^{y} is hydrogen, alkyl, alkenyl, alkynyl, cycloalkyl, heterocyclyl, aryl, heteroalkyl or heteroaryl; each of which may be optionally substituted, as defined herein. Examples of sulfonyl are methylsulfonyl, ethylsulfonyl, phenylsulfonyl and toluenesulfonyl.

“Sulfinyl” refers to the group —S(O)R^{y}, where R^{y} is hydrogen, alkyl, alkenyl, alkynyl, cycloalkyl, heterocyclyl, aryl, heteroalkyl or heteroaryl; each of which may be optionally substituted, as defined herein. Examples of sulfinyl are methylsulfinyl, ethylsulfinyl, phenylsulfinyl and toluenesulfinyl.

“Sulfonamido” refers to the groups —SO₂NR^{y}R^{z} and —NR^{y}SO₂R^{z}, where R^{y} and R^{z} are each independently hydrogen, alkyl, alkenyl, alkynyl, cycloalkyl, heterocyclyl, aryl, heteroalkyl or heteroaryl; each of which may be optionally substituted, as defined herein.

In some embodiments, the term “C₁₋₈alkoxy-C₁₋₈alkyl” refers to a radical of the formula: —C₁₋₈alkyl-O—C₁₋₈alkyl. In some embodiments, the term “C₁₋₈alkoxy-C₁₋₈alkyl-amino” refers to a radical of the formula: —NH—C₁₋₈alkyl-O—C₁₋₈alkyl. In some embodiments, the term “C₁₋₈alkoxy-C₂₋₈alkenyl” refers to a radical of the formula: —C₂₋₈alkenyl-O—C₁₋₈alkyl. In some embodiments, the term “C₁₋₈alkoxy-C₂₋₈alkynyl” refers to a radical of the formula: —C₂₋₈alkynyl-O—C₁₋₈alkyl. In some embodiments, the term “C₁₋₈alkoxy-carbonyl” refers to a radical of the formula: —C(O)—O—C₁₋₈alkyl. In some embodiments, the term “C₁₋₈alkoxy-carbonyl-C₁₋₈alkyl” refers to a radical of the formula: —C₁₋₈alkyl-C(O)—O—C₁₋₈alkyl. In some embodiments, the term “C₁₋₈alkoxy-carbonyl-amino” refers to a radical of the formula: —NH—C(O)—O—C₁₋₈alkyl. In some embodiments, the term “C₁₋₈alkoxy-carbonyl-amino-C₁₋₈alkyl” refers to a radical of the formula: —C₁₋₈alkyl-NH—C(O)—O—C₁₋₈alkyl. In some embodiments, the term “C₁₋₈alkoxy-imino-C₁₋₈alkyl” refers to a radical of the formula: —C₁₋₈alkyl(=N—O—C₁₋₈alkyl).

In some embodiments, the term “C₁₋₈alkyl-amino” refers to a radical of the formula: —NH—C₁₋₈alkyl. In some embodiments, the term “(C₁₋₈alkyl)₂-amino” refers to a radical of the formula: —N(C₁₋₈alkyl)₂. In some embodiments, the term “C₁₋₈alkyl-amino-C₁₋₈alkyl” refers to a radical of the formula: —C₁₋₈alkyl-NH—C₁₋₈alkyl. In some embodiments, the term “(C₁₋₈alkyl)₂-amino-C₁₋₈alkyl” refers to a radical of the formula: —C₁₋₈alkyl-N(C₁₋₈alkyl)₂. In some embodiments, the term “C₁₋₈alkyl-amino-C₁₋₈alkyl-amino” refers to a radical of the formula: —NH—C₁₋₈alkyl-NH—C₁₋₈alkyl. In some embodiments, the term “(C₁₋₈alkyl)₂-amino-C₁₋₈alkyl-amino” refers to a radical of the formula: —NH—C₁₋₈alkyl-N(C₁₋₈alkyl)₂. In some embodiments, the term “C₁₋₈alkyl-amino-carbonyl” refers to a radical of the formula: —C(O)—NH—C₁₋₈alkyl. In some embodiments, the term “(C₁₋₈alkyl)₂-amino-carbonyl” refers to a radical of the formula: —C(O)—N(C₁₋₈alkyl)₂. In some embodiments, the term “C₁₋₈alkyl-amino-carbonyl-amino” refers to a radical of the formula: —NH—C(O)—NH—C₁₋₈alkyl. In some embodiments, the term “(C₁₋₈alkyl)₂-amino-carbonyl-amino” refers to a radical of the formula: —NH—C(O)—N(C₁₋₈alkyl)₂.

In some embodiments, the term “C₁₋₈alkyl-amino-sulfonyl” refers to a radical of the formula: —SO₂—NH—C₁₋₈alkyl. In some embodiments, the term “(C₁₋₈alkyl)₂-amino-sulfonyl” refers to a radical of the formula: —SO₂—N(C₁₋₈alkyl)₂. In some embodiments, the term “C₁₋₈alkyl-amino-sulfonyl-amino” refers to a radical of the formula: —NH—SO₂—NH—C₁₋₈alkyl. In some embodiments, the term “(C₁₋₈alkyl)₂-amino-sulfonyl-amino” refers to a radical of the formula: —NH—SO₂—N(C₁₋₈alkyl)₂.

In some embodiments, the term “C₁₋₈alkyl-carbonyl” refers to a radical of the formula: —C(O)—C₁₋₈alkyl. In some embodiments, the term “C₁₋₈alkyl-carbonyl-amino” refers to a radical of the formula: —NH—C(O)—C₁₋₈alkyl. In some embodiments, the term “C₁₋₈alkyl-carbonyl-amino-C₁₋₈alkyl” refers to a radical of the formula: —C₁₋₈alkyl-NH—C(O)—C₁₋₈alkyl. In some embodiments, the term “C₁₋₈alkyl-carbonyl-oxy” refers to a radical of the formula: —O—C(O)—C₁₋₈alkyl. In some embodiments, the term “C₁₋₈alkyl-carbonyl-oxy-C₁₋₈ alkyl” refers to a radical of the formula: —C₁₋₈alkyl-O—C(O)—C₁₋₈alkyl.

In some embodiments, the term “C₁₋₈alkyl-sulfonyl” refers to a radical of the formula: —SO₂—C₁₋₈alkyl. In some embodiments, the term “C₁₋₈alkyl-thio” refers to a radical of the formula: —S—C₁₋₈alkyl.

In some embodiments, the term “amino” refers to a radical of the formula: —NH₂. In some embodiments, the term “amino-C₁₋₈alkyl” refers to a radical of the formula: —C₁₋₈alkyl-NH₂. n some embodiments, the term “amino-C₁₋₈alkyl-amino” refers to a radical of the formula: —NH—C₁₋₈alkyl-NH₂. In some embodiments, the term “amino-carbonyl” refers to a radical of the formula: —C(O)—NH₂. n some embodiments, the term “amino-carbonyl-amino” refers to a radical of the formula: —NH—C(O)—NH₂.

In some embodiments, the term “amino-sulfonyl” refers to a radical of the formula: —SO₂—NH₂. In some embodiments, the term “amino-sulfonyl-amino” refers to a radical of the formula: —NH—SO₂—NH₂.

In some embodiments, the term “aryl-C₁₋₈alkyl” refers to a radical of the formula: —C₁₋₈alkyl-aryl. In some embodiments, the term “aryl-C₁₋₈alkyl-amino” refers to a radical of the formula: —NH—C₁₋₈alkyl-aryl. In some embodiments, the term “aryl-amino” refers to a radical of the formula: —NH-aryl.

In some embodiments, the term “formyl” refers to a radical of the formula: —C(O)—H. In some embodiments, the term “formyl-oxyl” refers to a radical of the formula: —O—C(O)—H.”

In some embodiments, the term “halo-C₁₋₈alkoxy-carbonyl” refers to a radical of the formula: —C(O)—O—C₁₋₈haloalkyl. In some embodiments, the term “halo-C₁₋₈alkyl-carbonyl” refers to a radical of the formula: —C(O)—C₁₋₈haloalkyl. In some embodiments, the term “halo-C₁₋₈alkyl-sulfonyl” refers to a radical of the formula: —SO₂—C₁₋₈haloalkyl. In some embodiments, the term “halo-C₁₋₈alkyl-thio” refers to a radical of the formula: —S—C₁₋₈haloalkyl.

In some embodiments, the term “hydroxyl-C₁₋₈alkoxy” refers to a radical of the formula: —O—C₁₋₈alkyl-OH, wherein C₁₋₈alkyl may be partially or completely substituted where allowed by available valences with one or more hydroxyl radicals. In some embodiments, the term “hydroxyl-C₁₋₈alkyl” refers to a radical of the formula: —C₁₋₈alkyl-OH, wherein C₁₋₈alkyl may be partially or completely substituted where allowed by available valences with one or more hydroxyl radicals. In some embodiments, the term “hydroxyl-amino” refers to a radical of the formula: —NH—OH. In some embodiments, the term “hydroxyl-C₁₋₈alkyl-amino” refers to a radical of the formula: —NH—C₁₋₈alkyl-OH. In some embodiments, the term “hydroxyl-C₁₋₈alkyl-amino-C₁₋₈alkyl” refers to a radical of the formula: —C₁₋₈alkyl-NH—C₁₋₈alkyl-OH. In some embodiments, the term “hydroxyl-C₁₋₈alkyl-amino-C₁₋₈alkyl-amino” refers to a radical of the formula: —NH—C₁₋₈alkyl-NH—C₁₋₈ alkyl-OH. In some embodiments, the term “hydroxyl-imino-C₁₋₈alkyl” refers to a radical of the formula: —C₁₋₈alkyl(=N—OH).

In some embodiments, the term “imino” refers to a radical of the formula: ═NH. In some embodiments, the term “imino-C₁₋₈alkyl” refers to a radical of the formula: —C₁₋₈alkyl(=NH).

In some embodiments, the term “N-oxide” refers to a moiety of the formula:

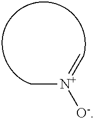

In some embodiments, the term “oxo” refers to a moiety of the formula:

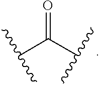 [SMILES: *C(*)=O]

In some embodiments, the term “P(O)(R₇)₂-amino” refers to a radical of the formulae: —NH—P(O)(—O—C₁₋₈alkyl)(OH) when R₇ is independently hydroxyl and (C₁₋₈alkoxy)ₙ, where n is 1; or, —NH—P(O)(OH)₂ when R₇ is hydroxyl; or, —NH—P(O)(—O—C₁₋₈alkyl)₂ when R₇ is (C₁₋₈alkoxy)ₙ, where n is 1.

The terms “optional” or “optionally” means that the subsequently described event or circumstance may or may not occur and that the description includes instances where said event or circumstance occurs and instances in which it does not. Also, the term “optionally substituted” refers to any one or more (e.g., 1 to 5, or 1 to 3) hydrogen atoms on the designated atom or group may or may not be replaced by a moiety other than hydrogen.

The term “substituted” used herein means any of the above groups (i.e., alkyl, alkenyl, alkynyl, alkylene, alkoxy, haloalkyl, haloalkoxy, cycloalkyl, aryl, heterocyclyl, heteroaryl, and/or heteroalkyl) wherein at least one (e.g., 1 to 5, or 1 to 3) hydrogen atom is replaced by a bond to a non-hydrogen atom such as, but not limited to alkyl, alkenyl, alkynyl, alkoxy, alkylthio, acyl, amido, amino, amidino, aryl, aralkyl, azido, carbamoyl, carboxyl, carboxyl ester, cyano, cycloalkyl, cycloalkylalkyl, guanidino, halo, haloalkyl, haloalkoxy, hydroxyalkyl, heteroalkyl, heteroaryl, heteroarylalkyl, heterocyclyl, heterocyclylalkyl, —NHNH₂, ═NNH₂, imino, imido, hydroxy, oxo, oxime, nitro, sulfonyl, sulfinyl, alkylsulfonyl, alkylsulfinyl, thiocyanate, —S(O)OH, —S(O)₂OH, sulfonamido, thiol, thioxo, N-oxide or —Si(R^{y})₃, wherein each R^{y} is independently hydrogen, alkyl, alkenyl, alkynyl, heteroalkyl, cycloalkyl, aryl, heteroaryl or heterocyclyl.

In certain embodiments, “substituted” includes any of the above alkyl, alkenyl, alkynyl, cycloalkyl, heterocyclyl, aryl or heteroaryl groups in which one or more (e.g., 1 to 5, or 1 to 3) hydrogen atoms are independently replaced with deuterium, halo, cyano, nitro, azido, oxo, alkyl, alkenyl, alkynyl, haloalkyl, cycloalkyl, heterocyclyl, aryl, heteroaryl, —NR^{g′}R^{h′}, —NR^{g′}C(═O)R^{h′}, —NR^{g′}C(═O)NR^{g′}R^{h′}, —NR^{g′}C(═O)OR^{h′}, —NR^{g′}S(═O)₁₋₂R^{h′}, —C(═O)R^{g′}, —C(═O)OR^{g′}, —OC(═O)OR^{g′}, —OC(═O)R^{g′}, —C(═O)NR^{g′}R^{h′}, —OC(═O)NR^{g′}R^{h′}, —OR^{g′}, —SR^{g′}, —S(═O)R^{g′}, —S(═O)₂R^{g′}, —OS(═O)₁₋₂R^{g′}, —S(═O)₁₋₂OR^{g′}, —NR^{g′}S(═O)₁₋₂NR^{g′}R^{h′}, ═NSO₂R^{g′}, ═NOR^{g′}, —S(═O)₁₋₂NR^{g′}R^{h′}, —SF₅, —SCF₃ or —OCF₃. In certain embodiments, “substituted” also means any of the above groups in which one or more (e.g., 1 to 5, or 1 to 3) hydrogen atoms are replaced with —C(═O)R^{g′}, —C(═O)OR^{g′}, —C(═O)NR^{g′}R^{h′}, —CH₂SO₂R^{g′}, or —CH₂SO₂NR^{g′}R^{h′}. In the foregoing, R^{g′} and R^{h′} are the same or different and independently hydrogen, alkyl, alkenyl, alkynyl, alkoxy, thioalkyl, aryl, aralkyl, cycloalkyl, cycloalkylalkyl, haloalkyl, heterocyclyl, heterocyclylalkyl, heteroaryl, and/or heteroarylalkyl. In certain embodiments, “substituted” also means any of the above groups in which one or more (e.g., 1 to 5, or 1 to 3) hydrogen atoms are replaced by a bond to an amino, cyano, hydroxyl, imino, nitro, oxo, thioxo, halo, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, and/or heteroarylalkyl, or two of R^{g′} and R^{h′} are taken together with the atoms to which they are attached to form a heterocyclyl ring optionally substituted with oxo, halo or alkyl optionally substituted with oxo, halo, amino, hydroxyl, or alkoxy.

Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein.

Any compound or structure given herein, is intended to represent unlabeled forms as well as isotopically labeled forms (isotopologues) of the compounds. These forms of compounds may also be referred to as and include “isotopically enriched analogs.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Various isotopically labeled compounds of the present disclosure, for example those into which radioactive isotopes such as ³H, ¹³C and ¹⁴C are incorporated, are provided. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The term “isotopically enriched analogs” includes “deuterated analogs” of compounds described herein in which one or more hydrogens is/are replaced by deuterium, such as a hydrogen on a carbon atom. Such compounds exhibit increased resistance to metabolism and are thus useful for increasing the half-life of any compound when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F, ³H, ¹¹C labeled compound may be useful for PET or SPECT or other imaging studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium. Further, in some embodiments, the corresponding deuterated analog is provided.

The term “hydrate” refers to the complex formed by the combining of a compound described herein and water.

A “solvate” refers to an association or complex of one or more solvent molecules and a compound of the disclosure. Examples of solvents that form solvates include, but are not limited to, water, isopropanol, ethanol, methanol, dimethylsulfoxide, ethylacetate, acetic acid and ethanolamine.

Some of the compounds exist as tautomers. Tautomers are in equilibrium with one another. For example, amide containing compounds may exist in equilibrium with imidic acid tautomers. Regardless of which tautomer is shown and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

The compounds of the disclosure, or their pharmaceutically acceptable salts include an asymmetric center and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high performance liquid chromatography (HPLC). When the compounds described herein contain olefinic double bonds or other centres of geometric asymmetry, and unless specified otherwise, it is intended that the compounds include both E and Z geometric isomers.

A “stereoisomer” refers to a compound made up of the same atoms bonded by the same bonds but having different three-dimensional structures, which are not interchangeable. The present disclosure contemplates various stereoisomers and mixtures thereof and includes “enantiomers,” which refers to two stereoisomers whose molecules are nonsuperimposable mirror images of one another.

“Diastereomers” are stereoisomers that have at least two asymmetric atoms, but which are not mirror-images of each other.

Relative centers of the compounds as depicted herein are indicated graphically using the “thick bond” style (bold or parallel lines) and absolute stereochemistry is depicted using wedge bonds (bold or parallel lines).

“Prodrugs” means any compound which releases an active parent drug according to a structure described herein in vivo when such prodrug is administered to a mammalian subject. Prodrugs of a compound described herein are prepared by modifying functional groups present in the compound described herein in such a way that the modifications may be cleaved in vivo to release the parent compound. Prodrugs may be prepared by modifying functional groups present in the compounds in such a way that the modifications are cleaved, either in routine manipulation or in vivo, to the parent compounds. Prodrugs include compounds described herein wherein a hydroxy, amino, carboxyl, or sulfhydryl group in a compound described herein is bonded to any group that may be cleaved in vivo to regenerate the free hydroxy, amino, or sulfhydryl group, respectively. Examples of prodrugs include, but are not limited to esters (e.g., acetate, formate and benzoate derivatives), amides, guanidines, carbamates (e.g., N,N-dimethylaminocarbonyl) of hydroxy functional groups in compounds described herein and the like. Preparation, selection and use of prodrugs is discussed in T. Higuchi and V. Stella, “Pro-drugs as Novel Delivery Systems,” Vol. 14 of the A.C.S. Symposium Series; “Design of Prodrugs,” ed. H. Bundgaard, Elsevier, 1985; and in Bioreversible Carriers in Drug Design, ed. Edward B. Roche, American Pharmaceutical Association and Pergamon Press, 1987, each of which are hereby incorporated by reference in their entirety.

“Pharmaceutically acceptable” or “physiologically acceptable” refer to compounds, salts, compositions, dosage forms and other materials which are useful in preparing a pharmaceutical composition that is suitable for human or veterinary pharmaceutical use.

The term “pharmaceutically acceptable salt” of a given compound refers to salts that retain the biological effectiveness and properties of the given compound, and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

As used herein, the term “administration” refers to introducing an agent into a patient. For example, a therapeutic amount can be administered to the patient, which can be determined by the treating physician, medical professional, or the like. In some embodiments, an oral route of administration is preferred. The related terms and phrases “administering” and “administration of,” when used in connection with a compound or tablet (and grammatical equivalents) refer both to direct administration, which may be administration to a patient by a medical professional or by self-administration by the patient, and/or to indirect administration, which may be the act of prescribing a drug. Administration entails delivery to the patient of the drug.

The term “dose” or “dosage” refers to the total amount of an active agent (e.g., compounds as described herein or a pharmaceutically acceptable salt thereof) administered to a patient in a single day (24-hour period). The desired dose can be administered once daily. In some embodiments, the desired dose may be administered in one, two, three, four or more sub-doses at appropriate intervals throughout the day, where the cumulative amount of the sub-doses equals the amount of the desired dose administered in a single day. The terms “dose” and “dosage” are used interchangeably herein.

As used herein, “therapeutically effective amount” or “therapeutic amount” refers to an amount of a drug or an agent (e.g., compounds as described herein or a pharmaceutically acceptable salt thereof) that when administered to a patient suffering from a condition, will have the intended therapeutic effect, e.g., alleviation, amelioration, palliation or elimination of one or more manifestations of the condition in the patient. The full therapeutic effect does not necessarily occur by administration of one dose, and can occur only after administration of a series of doses and can be administered in one dose form or multiples thereof. For example, 500 mg of the drug can be administered in a single 500 mg strength tablet or two 250 mg strength tablets. Thus, a therapeutically effective amount may be administered in one or more administrations.

As used herein, the term “patient” refers to a mammal, such as a human, bovine, rat, mouse, dog, monkey, ape, goat, sheep, cow, or deer. A patient as described herein can be a human.

As used herein, “treatment,” “treating,” and “treat” are defined as acting upon a disease, disorder, or condition with an agent to reduce or ameliorate the harmful or any other undesired effects of the disease, disorder, or condition and/or its symptoms. Treatment, as used herein, covers the treatment of a human patient, and includes: (a) reducing the risk of occurrence of the condition in a patient determined to be predisposed to the disease but not yet diagnosed as having the condition, (b) impeding the development of the condition, and/or (c) relieving the condition, i.e., causing regression of the condition and/or relieving one or more symptoms of the condition.

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The selected compounds may be further characterized to examine the safety or tolerance dosage in human or non-human subjects. Such properties may be examined using commonly known methods to those skilled in the art.

### Methods of Treatment

BMI1

BMI1 encodes a core component of the polycomb repressive complex 1 (PRC1) and is known to be involved in epigenetic regulation. It is contemplated herein that modulating BMI1 can activate embryonic or fetal hemoglobin expression, thereby treating sickle cell disease or β-thalassemia.

Provided herein are methods of treating a β-globinopathy comprising administering to the subject a therapeutically effective amount of a compound that modulates activity of BMI1 protein.

Provided herein are methods of treating a β-globinopathy comprising administering to the subject a pharmaceutical composition comprising a therapeutically effective amount of a compound that modulates activity of BMI1 protein and a pharmaceutically acceptable carrier.

In some embodiments, the β-globinopathy is sickle cell disease. In some embodiments, the β-globinopathy is β-thalassemia.

In some embodiments, a compound that modulates activity of BMI1 protein is a compound that inhibits activity of BMI1 protein (a BMI1 inhibitor). Non-limiting examples of a BMI1 inhibitor are as described herein.

In some embodiments, provided is a method for treating sickle cell disease in a subject in need thereof comprising administering to the subject a therapeutically effective amount of a compound that modulates activity of BMI1 protein.

In some embodiments, provided is a method for treating sickle cell disease in a subject in need thereof comprising administering to the subject a pharmaceutical composition comprising a therapeutically effective amount of a compound that modulates activity of BMI1 protein and a pharmaceutically acceptable carrier.

In some embodiments, provided is a method for treating β-thalassemia in a subject in need thereof comprising administering to the subject a therapeutically effective amount of a compound that modulates activity of BMI1 protein.

In some embodiments, provided is a method for treating β-thalassemia in a subject in need thereof comprising administering to the subject a pharmaceutical composition comprising a therapeutically effective amount of a compound that modulates activity of BMI1 protein and a pharmaceutically acceptable carrier.

In some embodiments, a compound that modulates activity of BMI1 protein is a compound that degrades the BMI1 protein.

In some embodiments, provided is a method for treating sickle cell disease in a subject in need thereof comprising administering to the subject a therapeutically effective amount of a compound that inhibits activity of BMI1 protein.

In some embodiments, provided is a method for treating sickle cell disease in a subject in need thereof comprising administering to the subject a pharmaceutical composition comprising a therapeutically effective amount of a compound that inhibits activity of BMI1 protein and a pharmaceutically acceptable carrier.

In some embodiments, provided is a method for treating β-thalassemia in a subject in need thereof comprising administering to the subject a therapeutically effective amount of a compound that inhibits activity of BMI1 protein.

In some embodiments, provided is a method for treating β-thalassemia in a subject in need thereof comprising administering to the subject a pharmaceutical composition comprising a therapeutically effective amount of a compound that inhibits activity of BMI1 protein and a pharmaceutically acceptable carrier.

Some embodiments provide for a method of treating sickle cell disease comprising administering to a subject in need thereof a therapeutically effective amount of a compound, or a pharmaceutical composition thereof, wherein the compound inhibits activity of BMI1 protein (a BMI1 inhibitor) to induce fetal hemoglobin expression and/or embryonic hemoglobin expression.

Some embodiments provide for a method of treating β-thalassemia comprising administering to a subject in need thereof a therapeutically effective amount of a compound, or a pharmaceutical composition thereof, wherein the compound inhibits activity of BMI1 protein (a BMI1 inhibitor) to induce fetal hemoglobin expression and/or embryonic hemoglobin expression.

In some embodiments, methods described herein further comprise increasing embryonic hemoglobin expression.

In some embodiments, methods described herein further comprise increasing fetal hemoglobin expression.

In some embodiments, methods described herein comprise administering a compound that modulates activity of a BMI1 protein as a pharmaceutically acceptable salt of such compound.

In some embodiments, methods described herein comprise administering a compound that inhibits activity of a BMI1 protein as a pharmaceutically acceptable salt of such compound.

Some embodiments provide for a method of treating a β-globinopathy comprising administering to a subject in need thereof a therapeutically effective amount of PTC596, or a pharmaceutical composition thereof.

Some embodiments provide for a method of treating sickle cell disease comprising administering to a subject in need thereof a therapeutically effective amount of PTC596, or a pharmaceutical composition thereof.

Some embodiments provide for a method of treating β-thalassemia comprising administering to a subject in need thereof a therapeutically effective amount of a PTC596, or a pharmaceutical composition thereof.

MEN1 and KMT2A

Mixed Lineage Leukemia (MLL) complex is known to induce transcriptional activation and is also known to induce methylation of H3K4 and acetylation of H4K16. Major complex components include KMT2A (MLL1), MEN1 (MENIN), LEDGF, RBBP5, ASH2L, WDR5, and DPY30.

It is contemplated herein that modulating the activity of MLL complex, MEN1, or KMT2A or modulating the interaction between MEN1 and KMT2A can activate fetal and/or embryonic hemoglobin expression, thereby treating sickle cell disease or β-thalassemia.

It is further contemplated herein that MENIN inhibitors, which are compounds that can inhibit MLL complex by blocking the interaction between MEN1 and KMT2A, activate fetal and/or embryonic hemoglobin expression, thereby treating sickle cell disease or β-thalassemia.

Provided herein are methods of treating a β-globinopathy comprising administering to the subject a therapeutically effective amount of a compound that modulates activity of MLL complex, MEN1, or KMT2A or modulates the interaction between MEN1 and KMT2A.

Provided herein are methods of treating a β-globinopathy comprising administering to the subject a pharmaceutical composition comprising a therapeutically effective amount of a compound that modulates activity of MLL complex, MEN1, or KMT2A or modulates the interaction between MEN1 and KMT2A and a pharmaceutically acceptable carrier.

Provided herein are methods of treating a β-globinopathy comprising administering to the subject a therapeutically effective amount of a compound that modulates the interaction between MEN1 and KMT2A.

Provided herein are methods of treating a β-globinopathy comprising administering to the subject a pharmaceutical composition comprising a therapeutically effective amount of a compound that modulates modulates the interaction between MEN1 and KMT2A and a pharmaceutically acceptable carrier.

Provided herein are methods of treating a β-globinopathy comprising administering to the subject a therapeutically effective amount of a compound that modulates activity of MEN1.

Provided herein are methods of treating a β-globinopathy comprising administering to the subject a pharmaceutical composition comprising a therapeutically effective amount of a compound that modulates modulates activity of MEN1 and a pharmaceutically acceptable carrier.

Provided herein are methods of treating a β-globinopathy comprising administering to the subject a therapeutically effective amount of a compound that modulates activity of KMT2A.

Provided herein are methods of treating a β-globinopathy comprising administering to the subject a pharmaceutical composition comprising a therapeutically effective amount of a compound that modulates modulates activity of KMT2A and a pharmaceutically acceptable carrier.

In some embodiments, the β-globinopathy is sickle cell disease. In some embodiments, the β-globinopathy is β-thalassemia.

In some embodiments, provided is a method for treating sickle cell disease in a subject in need thereof comprising administering to the subject a therapeutically effective amount of a compound that modulates the activity of Mixed Lineage Leukemia (MLL) complex.

In some embodiments, provided is a method for treating sickle cell disease in a subject in need thereof comprising administering to the subject a pharmaceutical composition comprising a therapeutically effective amount of a compound that modulates the activity of MLL complex and a pharmaceutically acceptable carrier.

In some embodiments, provided is a method for treating β-thalassemia in a subject in need thereof comprising administering to the subject a therapeutically effective amount of a compound that modulates the activity of MLL complex.

In some embodiments, provided is a method for treating β-thalassemia in a subject in need thereof comprising administering to the subject a pharmaceutical composition comprising a therapeutically effective amount of a compound that modulates the activity of MLL complex and a pharmaceutically acceptable carrier.

In some embodiments, a compound that modulates the activity of Mixed Lineage Leukemia (MLL) complex is a compound that inhibits the MLL complex.

In some embodiments, provided is a method for treating sickle cell disease in a subject in need thereof comprising administering to the subject a therapeutically effective amount of a compound that inhibits activity of MLL complex.

In some embodiments, provided is a method for treating sickle cell disease in a subject in need thereof comprising administering to the subject a pharmaceutical composition comprising a therapeutically effective amount of a compound that inhibits activity of MLL complex and a pharmaceutically acceptable carrier.

In some embodiments, provided is a method for treating β-thalassemia in a subject in need thereof comprising administering to the subject a therapeutically effective amount of a compound that inhibits activity of MLL complex.

In some embodiments, provided is a method for treating β-thalassemia in a subject in need thereof comprising administering to the subject a pharmaceutical composition comprising a therapeutically effective amount of a compound that inhibits activity of MLL complex and a pharmaceutically acceptable carrier.

In some embodiments, a compound that inhibits the activity of MLL complex is a compound that inhibits activity of MEN1 protein.

In some embodiments, a compound that inhibits the activity of MLL complex is a compound that inhibits activity of KMT2A protein.

In some embodiments, a compound that inhibits the MLL complex is a compound that inhibits interaction between MEN1 protein and KMT2A protein (a MENIN inhibitor or MENIN-MLL inhibitor).

Some embodiments provide for a method of treating sickle cell disease comprising administering to a subject in need thereof a therapeutically effective amount of a compound, or a pharmaceutical composition thereof, wherein the compound inhibits interaction between MEN1 protein and KMT2A protein (a MENIN inhibitor or MENIN-MLL inhibitor) to induce fetal hemoglobin expression and/or embryonic hemoglobin expression.

Some embodiments provide for a method of treating β-thalassemia comprising administering to a subject in need thereof a therapeutically effective amount of a compound, or a pharmaceutical composition thereof, wherein the compound inhibits interaction between MEN1 protein and KMT2A protein (a MENIN inhibitor or MENIN-MLL inhibitor) to induce fetal hemoglobin expression and/or embryonic hemoglobin expression.

In some embodiments, methods described herein further comprise increasing embryonic hemoglobin expression.

In some embodiments, methods described herein further comprise increasing fetal hemoglobin expression.

In some embodiments, methods described herein comprise administering a pharmaceutically acceptable salt of a compound as described herein.

Some embodiments provide for a method of treating a β-globinopathy comprising administering to a subject in need thereof a therapeutically effective amount of MI-3454, or a pharmaceutical composition thereof.

Some embodiments provide for a method of treating sickle cell disease comprising administering to a subject in need thereof a therapeutically effective amount of MI-3454, or a pharmaceutical composition thereof.

Some embodiments provide for a method of treating β-thalassemia comprising administering to a subject in need thereof a therapeutically effective amount of a MI-3454, or a pharmaceutical composition thereof.

### Compounds and Compositions Thereof

In some embodiments, a compound that modulates activity of BMI1 protein is a compound that degrades the BMI1 protein. In some embodiments, a compound that modulates activity of BMI1 protein is a compound that inhibits activity of BMI1 protein (a BMI1 inhibitor).

In some embodiments, a compound that inhibits activity of BMI1 protein (a BMI1 inhibitor) induces embryonic hemoglobin expression or fetal hemoglobin expression. In some embodiments, a compound that inhibits activity of BMI1 protein is a compound that accelerates BMI1 degradation. In some embodiments, the compound that inhibits activity of BMI1 protein is PTC596.

In some embodiments, the BMI1 inhibitor is a compound having an amine-substituted reverse pyrimidine ring.

In some embodiments, the BMI1 inhibitor is a compound of Formula (I):

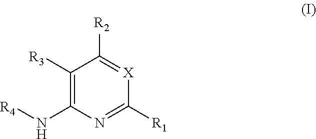

or a free acid, free base, salt, ester, hydrate, solvate, chelate, clathrate, isotopologue, stereoisomer, racemate, enantiomer, diastereomer, and tautomer thereof, wherein:

R₁ is bicyclic heteroaryl or bicyclic heterocyclyl substituted on a carbon atom ring member with one, two, three, or four R₅ substituents, or on a nitrogen atom ring member with an oxygen atom substituent to form an N-oxide;

X is N or N substituted with an oxygen atom substituent to form an N-oxide;

R₂ is amino;

R₃ is hydrogen, cyano, halo, C₁₋₈alkyl, amino, C₁₋₈alkyl-amino, or (C₁₋₈alkyl)₂-amino;

R₄ is phenyl, optionally substituted with one, two, three, or four R₆ substituents;

R₅ is independently selected from the group consisting of cyano, halo, hydroxyl, nitro, oxo, C₁₋₈alkyl, cyano-C₁₋₈alkyl, halo-C₁₋₈alkyl, hydroxyl-C₁₋₈alkyl, C₁₋₈alkoxy, C₁₋₈alkoxy-C₁₋₈alkyl, halo-C₁₋₈alkoxy, C₂₋₈alkenyl, C₁₋₈alkoxy-C₂₋₈alkenyl, C₂₋₈alkynyl, C₁₋₈alkoxy-C₂₋₈alkynyl, carboxyl, amino, C₁₋₈alkyl-amino, (C₁₋₈alkyl)₂-amino, amino-C₁₋₈alkyl, C₁₋₈alkyl-amino-C₁₋₈alkyl, (C₁₋₈alkyl)₂-amino-C₁₋₈alkyl, hydroxyl-C₁₋₈alkyl-amino, hydroxyl-C₁₋₈alkyl-amino-C₁₋₈alkyl, hydroxyl-C₁₋₈alkyl-amino-C₁₋₈alkyl-amino, C₁₋₈alkyl-thio, C₁₋₈ alkyl-carbonyl, C₁₋₈alkyl-carbonyl-amino, C₁₋₈alkyl-carbonyl-oxy, C₁₋₈alkyl-carbonyl-oxy-C₁₋₈alkyl, C₁₋₈ alkoxy-carbonyl, C₁₋₈alkoxy-carbonyl-C₁₋₈alkyl, C₁₋₈alkoxy-carbonyl-amino, C₁₋₈alkyl-sulfonyl, C₃₋₁₄cycloalkyl, aryl, aryl-C₁₋₈alkyl, aryl-amino, aryl-C₁₋₈alkyl-amino, heteroaryl, heteroaryl-C₁₋₈alkyl, and heterocyclyl, wherein C₃₋₁₄cycloalkyl, aryl, heteroaryl, or heterocyclyl and the aryl and heteroaryl portions of aryl-C₁₋₈alkyl, aryl-amino, aryl-C₁₋₈alky-amino, and heteroaryl-C₁₋₈alkyl are each optionally substituted with one, two, three, or four halo, C₁₋₈alkyl, halo-C₁₋₈alkyl, hydroxyl-C₁₋₈alkyl, C₁₋₈alkoxy, halo-C₁₋₈alkoxy, hydroxyl-C₁₋₈alkoxy, or carboxyl substituents;

R₆ is independently selected from the group consisting of cyano, halo, nitro, C₁₋₈alkyl, halo-C₁₋₈alkyl, hydroxyl-C₁₋₈alkyl, C₁₋₈alkoxy, halo-C₁₋₈alkoxy, C₂₋₈alkenyl, C₁₋₈alkoxy-C₂₋₈alkenyl, C₂₋₈alkynyl, C₁₋₈alkoxy-C₂₋₈alkynyl, carboxyl, formyl, formyl-oxy, C₁₋₈alkyl-carbonyl, halo-C₁₋₈alkyl-carbonyl, C₁₋₈alkyl-thio, halo-C₁₋₈alkyl-thio, amino, C₁₋₈alkyl-amino, (C₁₋₈alkyl)₂-amino, C₁₋₈alkyl-carbonyl, C₁₋₈alkyl-carbonyl-oxy, C₁₋₈alkyl-carbonyl-oxy-C₁₋₈alkyl, C₁₋₈alkoxy-carbonyl, halo-C₁₋₈alkoxy-carbonyl, C₁₋₈alkoxy-carbonyl-C₁₋₈alkyl, C₁₋₈alkoxy-carbonyl-amino, C₁₋₈alkoxy-carbonyl-amino-C₁₋₈alkyl, amino-carbonyl, C₁₋₈alkyl-amino-carbonyl, (C₁₋₈alkyl)₂-amino-carbonyl, C₁₋₈alkyl-carbonyl-amino, C₁₋₈alkyl-carbonyl-amino-C₁₋₈alkyl, amino-C₁₋₈alkyl, C₁₋₈alkyl-amino-C₁₋₈alkyl, (C₁₋₈alkyl)₂-amino-C₁₋₈alkyl, amino-C₁₋₈alkyl-amino, C₁₋₈alkyl-amino-C₁₋₈alkyl-amino, (C₁₋₈alkyl)₂-amino-C₁₋₈alkyl-amino, hydroxyl-C₁₋₈alkyl-amino, hydroxyl-C₁₋₈alkyl-amino-C₁₋₈alkyl, hydroxyl-C₁₋₈alkyl-amino-C₁₋₈alkyl-amino, imino-C₁₋₈alkyl, hydroxyl-imino-C₁₋₈alkyl, C₁₋₈alkoxy-imino-C₁₋₈alkyl, C₁₋₈alkyl-sulfonyl, halo-C₁₋₈alkyl-sulfonyl, amino-sulfonyl, C₁₋₈alkyl-amino-sulfonyl, (C₁₋₈alkyl)₂-amino-sulfonyl, B(OR₈)₂, C₃₋₁₄cycloalkyl, heterocyclyl, aryl, and heteroaryl, wherein C₃₋₁₄cycloalkyl, heterocyclyl, aryl, and heteroaryl are each optionally substituted with one, two, three or four halo or C₁₋₈alkyl substituents; and

R₈ is independently hydrogen or C₁₋₈alkyl.

Such compounds can be made according to methods known in the art, such as those described in U.S. Pat. No. 10,428,050, which is hereby incorporated by reference in its entirety.

In some embodiments, the BMI1 inhibitor is a compound of Formula (I-a):

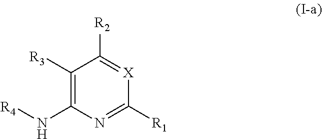

or a free acid, free base, salt, ester, hydrate, solvate, chelate, clathrate, isotopologue, stereoisomer, racemate, enantiomer, diastereomer, and tautomer thereof, wherein:

R₁ is bicyclic heteroaryl substituted on a carbon atom ring member with one or two R₅ substituents;

X is N;

R₂ is amino;

R₃ is hydrogen, halo, C₁₋₈alkyl, amino, C₁₋₈alkyl-amino, or (C₁₋₈alkyl)₂-amino;

R₄ is phenyl, optionally substituted with one or two R₆ substituents;

R₅ is independently selected from the group consisting of halo, C₁₋₈alkyl, or halo-C₁₋₈alkyl;

R₆ is independently selected from the group consisting of halo, C₁₋₈alkyl, or halo-C₁₋₈alkyl.

In some embodiments, the BMI1 inhibitor is a compound having an amine-substituted thiazole ring.

In some embodiments, the BMI1 inhibitor is a compound of Formula (II):

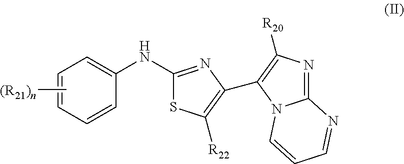

or a pharmaceutically acceptable salt thereof, wherein:

R₂₀ is H or methyl;

each R₂₁ is independently methyl or —OCH₃; or

two instances of R₂₁ join together to form a 5-membered or 6-membered heterocycloalkyl ring;

R₂₂ is H or methyl; and

n is 1 or 2.

Such compounds can be made according to methods known in the art, such as those described in Bartucci et al., Target Oncol. 2017, 12(4), 449-462, which is hereby incorporated by reference in its entirety.

In some embodiments, the BMI1 inhibitor is RU-A1, QW24, PRT4165, PTC-209, PTC-028, PTC596, or a pharmaceutically acceptable salt thereof. In some embodiments, the BMI1 inhibitor is PTC596.

Non-limiting examples of BMI1 inhibitors are shown below:

| Name | Structure |
|---|---|
| RU-A1 | 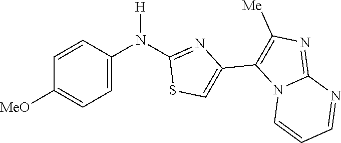 |
| QW24 | 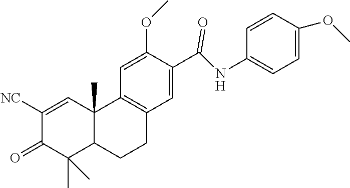 [SMILES: COc1ccc(NC(=O)c2cc3c(cc2OC)[C@@]2(C)C=C(C#N)C(=O)C(C)(C)C2CC3)cc1] |
| PRT4165 | 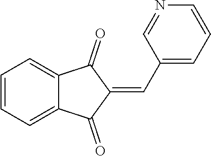 [SMILES: O=C1C(=Cc2cccnc2)C(=O)c2ccccc21] |
| PTC-209 | 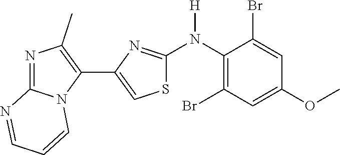 [SMILES: COc1cc(Br)c(Nc2nc(-c3c(C)nc4ncccn34)cs2)c(Br)c1] |
| PTC-028 | 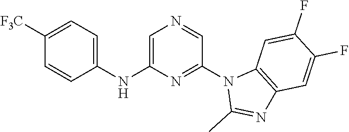 [SMILES: Cc1nc2cc(F)c(F)cc2n1-c1cncc(Nc2ccc(C(F)(F)F)cc2)n1] |
| PTC596 | 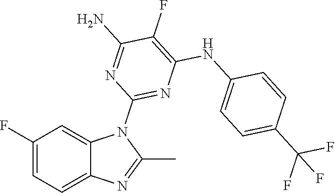 [SMILES: Cc1nc2ccc(F)cc2n1-c1nc(N)c(F)c(Nc2ccc(C(F)(F)F)cc2)n1] |

Such compounds are commercially available or can be made according to methods known in the art.

In some embodiments, a BMI1 inhibitor may be a gene editing system disrupting the BMI1 genomic sequence. In some embodiments, the BMI1 inhibitor is one or more components of a gene editing system targeting one or more sites within a gene encoding BMI1 or a regulatory element thereof, a nucleic acid molecule encoding the one or more components of the gene editing system, or a combination thereof. In some embodiments, the BMI1 inhibitor is a gene editing system, and wherein the gene editing system is selected from the group consisting of CRISPR/Cas9, CRISPR/Cas13, a zinc finger nuclease system, a TALEN system, and a meganuclease system. In some embodiments, the gene editing system is a CRISPR/Cas9 system. In some embodiments, the gene editing system is a CRISPR/Cas13 system. In some embodiments, the gene editing system is a zinc finger nuclease system. In some embodiments, the gene editing system is a TALEN system. In some embodiments, the gene editing system is a meganuclease system. In some embodiments, the BMI1 inhibitor comprises a guide RNA molecule comprising a tracr and a crRNA. In some embodiments, the crRNA comprises a targeting domain that is complementary with a target sequence of BMI1.

In some embodiments, a compound that modulates the activity of Mixed Lineage Leukemia (MLL) complex is a compound that inhibits the MLL complex.

In some embodiments, a compound that modulates the activity of MLL complex is a compound that modulates the interaction between MEN1 and KMT2A.

In some embodiments, a compound that modulates the interaction between MEN1 and KMT2A is a compound that inhibits the interaction between MEN1 and KMT2A (a MENIN inhibitor or MENIN-MLL inhibitor). In some embodiments, a compound that inhibits interaction between MEN1 protein and KMT2A protein is MI-3454.

In some embodiments, a compound that inhibits the interaction between MEN1 and KMT2A is a compound that binds to the MLL1 binding site on menin.

In some embodiments, the MENIN inhibitor is a compound having a thienopyrimidine scaffold. In some embodiments, the MENIN inhibitor is a compound having a hydroxymethylpiperidine scaffold. In some embodiments, MENIN inhibitor is a peptidomimetic inhibitor. Non-limiting examples of such compounds are known in the art, such as those described in Cierpicki et al., Future Med Chem., 2014, 6(4), 447-462, which is hereby incorporated by reference in its entirety. In some embodiments, the MENIN inhibitor is MI-1, MI-2, MI-2-2, MLS001171971-01, ML227, or MCP-1.

In some embodiments, the MENIN inhibitor is a compound of Formula (III):

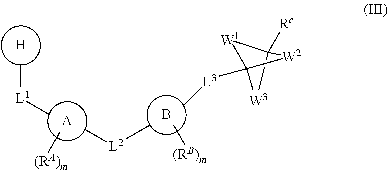

or a pharmaceutically acceptable salt, isotopic form, or prodrug thereof, wherein:
ring H is selected from C₅₋₁₂ carbocycle and 5- to 12-membered heterocycle, each of which is optionally substituted with one or more R⁵⁰;
ring A is selected from bond, C₃₋₁₂ carbocycle and 3- to 12-membered heterocycle;
ring B is selected from C₃₋₁₂ carbocycle and 3- to 12-membered heterocycle;
L¹, L², and L³ are each independently selected from bond, —O—, —S—, —N(R⁵¹)—, —N(R⁵¹)CH₂—, —C(O)—, —C(O)O—, —OC(O)—, —OC(O)O—, —C(O)N(R⁵¹)—, —C(O)N(R⁵¹)C(O)—, —C(O)N(R⁵¹)C(O)N(R⁵¹)—, —N(R⁵¹)C(O)—, —N(R⁵¹)C(O)N(R⁵¹)—, —N(R⁵¹)C(O)O—, —OC(O)N(R⁵¹)—, —C(NR⁵¹)—, —N(R⁵¹)C(NR⁵¹)—, —C(NR⁵¹)N(R⁵¹)—, —N(R⁵¹)C(NR¹)N(R⁵¹)—, —S(O)₂—, —OS(O)—, —S(O)O—, —S(O)—, —OS(O)₂—, —S(O)₂O—, —N(R⁵¹)S(O)₂—, —S(O)₂N(R⁵¹)—, —N(R⁵¹)S(O)—, —S(O)N(R⁵¹)—, —N(R⁵¹)S(O)₂N(R⁵¹)—, —N(R⁵¹)S(O)N(R⁵¹)—; alkylene, alkenylene, alkynylene, heteroalkylene, heteroalkenylene, and heteroalkynylene, each of which is optionally substituted with one or more R⁵⁰;

R^{A}, R^{B}, and R^{C} are each independently selected at each occurrence from R⁵⁰, or two R^{A} groups or two R^{B} groups attached to the same atom or different atoms can together optionally form a bridge or ring;

m and n are each independently an integer from 0 to 6;

W is C₁₋₄ alkylene, optionally substituted with one or more R⁵⁰;

W² is selected from a bond; and C₁₋₄ alkylene, optionally substituted with one or more R⁵⁰;

W³ is selected from absent; and C₁₋₄ alkylene, optionally substituted with one or more R⁵⁰;

R⁵⁰ is independently selected at each occurrence from:
halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, ═O, ═S, ═N(R⁵²);
C₁-C₁₀ alkyl, C₂₋₁₀ alkenyl, and C₂₋₁₀ alkynyl, each of which is independently optionally substituted at each occurrence with one or more substituents selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, ═O, ═S, ═N(R⁵²), C₃₋₁₂ carbocycle, and 3- to 12-membered heterocycle; and
C₃₋₁₂ carbocycle and 3- to 12-membered heterocycle,
wherein each C₃₋₁₂ carbocycle and 3- to 12-membered heterocycle in R⁵⁰ is independently optionally substituted with one or more substituents selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵²—, —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, ═O, ═S, ═N(R⁵²), C₁₋₆ alkyl, C₁₋₆ haloalkyl, C₂₋₆ alkenyl, and C₂₋₆ alkynyl;

R⁵¹ is independently selected at each occurrence from:
hydrogen, —C(O)R⁵², —C(O)OR⁵², —C(O)N(R⁵²)₂, —C(O)NR⁵³R⁵⁴; C₁₋₆ alkyl, C₂₋₆ alkenyl, and C₂₋₆ alkynyl, each of which is independently optionally substituted at each occurrence with one or more substituents selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, ═O, ═S, ═N(R⁵²), C₃₋₁₂ carbocycle, and 3- to 12-membered heterocycle; and
C₃₋₁₂ carbocycle and 3- to 12-membered heterocycle,
wherein each C₃₋₁₂ carbocycle and 3- to 12-membered heterocycle in R⁵¹ is independently optionally substituted with one or more substituents selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵²R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R^{S2})₂, ═O, ═S, ═N(R⁵²), C₁₋₆ alkyl, C₁₋₆ haloalkyl, C₂₋₆ alkenyl, and C₂₋₆ alkynyl;

R⁵² is independently selected at each occurrence from hydrogen; and C₁₋₂₀ alkyl, C₂₋₂₀ alkenyl, C₂₋₂₀ alkynyl, 2- to 6-membered heteroalkyl, C₃₋₁₂ carbocycle, and 3- to 12-membered heterocycle, each of which is optionally substituted by halogen, —CN, —NO₂, —NH₂, —NHCH₃, —NHCH₂CH₃, ═O, —OH, —OCH₃, —OCH₂CH₃, C₃₋₁₂ carbocycle, or 3- to 6-membered heterocycle; and

R⁵³ and R⁵⁴ are taken together with the nitrogen atom to which they are attached to form a heterocycle, optionally substituted with one or more R⁵⁰,

wherein for a compound or salt of Formula (I), when W³ is absent:

W¹ is C, alkylene, W² is a bond, and L³ is not a bond;

W¹ is C₂₋₄ alkylene and W² is a bond; or

W¹ and W² are each C₁ alkylene and L³ is not a bond, wherein each C₁ alkylene is independently optionally substituted with one or more R⁵⁰.

In some embodiments, the MENIN inhibitor is a compound of Formula (III-a):

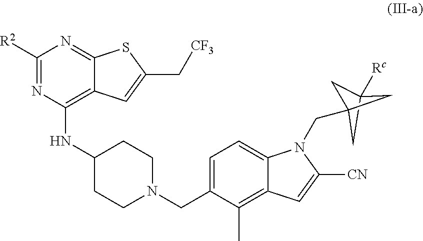

or a pharmaceutically acceptable salt, isotopic form, or prodrug thereof, wherein:

R^{C} is R⁵⁰;

R² is hydrogen or R⁵⁰;

R⁵⁰ is independently selected at each occurrence from:
halogen, —NO, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂;
C₁-C₁₀ alkyl, C₂₋₁₀ alkenyl, and C₂₋₁₀ alkynyl, each of which is independently optionally substituted at each occurrence with one or more substituents selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, ═O, ═S, ═N(R⁵²), C₃₋₁₂ carbocycle, and 3- to 12-membered heterocycle; and
C₃₋₁₂ carbocycle and 3- to 12-membered heterocycle,
wherein each C₃₋₁₂ carbocycle and 3- to 12-membered heterocycle in R⁵⁰ is independently optionally substituted with one or more substituents selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵²—, —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, ═O, ═S, ═N(R⁵²), C₁₋₆ alkyl, C₁₋₆ haloalkyl, C₂₋₆ alkenyl, and C₂₋₆ alkynyl;

R⁵² is independently selected at each occurrence from hydrogen; and C₁₋₂₀ alkyl, C₂₋₂₀ alkenyl, C₂₋₂₀ alkynyl, 2- to 6-membered heteroalkyl, C₃₋₁₂ carbocycle, and 3- to 12-membered heterocycle, each of which is optionally substituted by halogen, —CN, —NO₂, —NH₂, —NHCH₃, —NHCH₂CH₃, ═O, —OH, —OCH₃, —OCH₂CH₃, C₃₋₁₂ carbocycle, or 3- to 6-membered heterocycle; and

R⁵³ and R⁵⁴ are taken together with the nitrogen atom to which they are attached to form a heterocycle.

In some embodiments, R^{c} is —NR⁵²C(O)R⁵², and R² is hydrogen, —N(R⁵²)₂, or C₁₋₃alkyl.

Such compounds of Formula (III) and (III-a) can be made according to methods known in the art, such as those described in U.S. Pat. No. 10,752,639, which is hereby incorporated by reference in its entirety.

In some embodiments, the MENIN inhibitor is MI-503, MI-1481, or MI-3454. In some embodiments, the MENIN inhibitor is MI-3454.

In some embodiments, the MENIN inhibitor is a compound of Formula (IV):

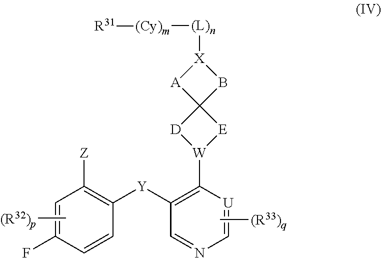

or a pharmaceutically acceptable salt thereof, wherein:

A, B, D, and E are each independently selected from —C(R^{A1})(R^{A2})—, —C(R^{A1})(R^{A2})—C(R^{A1})(R^{A2})—, —C(R^{A1})(R^{A2})—O—, —C(R^{A1})(R^{A2})—NR^{S3}—, —C(═O)—, —C(R^{A1})(R^{A2})—C(═O)—, and —N═C(NH₂)— wherein no more than one of A, B, D, and E is —C(R^{A1})(R^{A2})—O—, —C(R^{A1})(R^{A2})—NR^{A3}—, —C(R^{A1})(R^{A2})—C(═O)—, —C(═O)—, or —N═C(NH₂)—;

U is N or CR^{U}, wherein R^{U} is H, halo, CN, OH, C₁₋₄ alkyl, C₁₋₄ alkoxy, amino, C₁₋₄ alkyl amino, or C₂₋₈ dialkylamino;

W is N or CR^{W}, wherein R^{W} is H, halo, CN, OH, C₁₋₄ alkyl, C₁₋₄ alkoxy, amino, C₁₋₄ alkyl amino, or C₂₋₈ dialkylamino;

X is N or CR^{X}, wherein R^{X} is H, halo, CN, OH, C₁₋₄ alkyl, C₁₋₄ alkoxy, amino, C₁₋₄ alkyl amino, or C₂₋₈ dialkylamino, wherein when X is N, the atom of L, that is directly bonded with X is other than N, O, or S;

L is selected from —C₁₋₆ alkylene- and —(C₁₋₄ alkylene)ₐ-Q-(C₁₋₄ alkylene)_{b}-, wherein the C₁₋₆ alkylene group and any C₁₋₄ alkylene group of the —(C₁₋₄ alkylene)ₐ-Q-(C₁₋₄ alkylene)_{b}- group is optionally substituted with 1, 2, or 3 substituents independently selected from halo, CN, OH, C₁₋₃ alkyl, C₁₋₃ alkoxy, C₁₋₃ haloalkyl, C₁₋₃ haloalkoxy, amino, C₁₋₃ alkylamino, and di(C₁₋₃ alkyl)amino;

Q is —O—, —S—, —S(═O)—, —S(═O)₂—, —C(═O)—, —C(═O)NR^{q1}—, —C(═O)O—, —OC(═O)NR^{q1}—, —NR^{q1}—, —NR^{q1}C(═O)O—, —NR^{q1}C(═O)NR^{q1}—, —S(═O)₂NR^{q1}—, —C(═NR^{q2})—, or —C(═NR^{q2})—NR^{q1}—, wherein each R^{q1} is independently selected from H or C₁₋₆ alkyl, and wherein each R^{q2} is independently selected from H, C₁₋₆ alkyl, and CN;

Cy is a linking C₆₋₁₄ aryl, C₃₋₁₈ is cycloalkyl, 5-16 membered heteroaryl, or 4-18 membered heterocycloalkyl group, each of which is optionally substituted with 1, 2, 3, or 4 substituents independently selected from R^{Cy};

each R^{Cy} is independently selected from halo, C₁₋₆ alkyl, C₁₋₄ haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted by 1, 2, 3, or 4 substituents independently selected from CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

R³¹ is H, Cy¹, halo, C₁₋₆ alkyl, C₁₋₄ haloalkyl, C₁₋₄ cyanoalkyl, C₂₋₆ alkenyl, C₂₋₆ alkynyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{e2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2} and S(O)₂NR^{c2}R^{d2}, wherein said C₁₋₆ alkyl, C₂₋₆ alkenyl, and C₂₋₆ alkynyl are each optionally substituted by 1, 2, 3, or 4 substituents independently selected from halo, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{a2}R^{d2}, NR^{a2}R^{d2}, NR^{a2}C(O)R^{b2}, NR^{a2}C(O)OR^{a2}, NR^{a2}C(O)NR^{a2}R^{d2}, NR^{a2}S(O)R^{b2}, NR^{a2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};

Y is O, S, CR^{Y1}R^{Y2} or NR^{Y3}, wherein R^{Y1}, R^{Y2}, and R^{Y3} are each independently selected from H and C₁₋₄ alkyl;

Z is Cy², halo, C₁₋₆ alkyl, C₁₋₄ haloalkyl, C₁₋₄ cyanoalkyl, C₂₋₆ alkenyl, C₂₋₆ alkynyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{e3}C(O)R^{b3}, NR^{c3}C(O)OR^{a3}, NR^{e3}C(O)NR^{c3}R^{d3}, NR^{e3}S(O)R^{b3}, NR^{c3}S(O)₂R^{b3}, NR^{e3}S(O)₂NR^{c3}R^{d3}, S(O)R^{b3}, S(O)NR^{c3}R^{d3}, S(O)₂R^{b3}, S(O)₂NR^{c3}R^{d3}, and P(O)R^{c3}R^{d3} wherein said C₁₋₆ alkyl, C₂₋₆ alkenyl, and C₂₋₆ alkynyl are each optionally substituted by 1, 2, 3, or 4 substituents independently selected from Cy², halo, CN, NO₂, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};

each R³² and R³³ is independently selected from H, halo, C₁₋₆ alkyl, C₁₋₄ haloalkyl, C₁₋₄cyanoalkyl, C₂₋₆ alkenyl, C₂₋₆ alkynyl, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, C(═NR^{e4})NR^{c4}R^{d4}, NR^{c4}C(═NR^{e4})NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}, wherein said C₁₋₆ alkyl, C₂₋₆ alkenyl, and C₂₋₆ alkynyl are each optionally substituted by 1, 2, 3, or 4 substituents independently selected from halo, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, C(═NR^{e4})NR^{c4}R^{d4}, NR^{c4}C(═NR^{e4})NR^{c4}R^{d4}, NR^{c4}R^{d4}, NR^{e4}C(O)R^{b4}, NR^{c4}C(O)OR^{a4}, NR^{c4}C(O)NR^{e4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{e4}R^{d4}, S(O)₂R^{b4}, and S(O)₂NR^{c4}R^{d4};

each R^{A1} is independently selected from H, halo, C₁₋₄ alkyl, C₁₋₄ alkoxy, C₁₋₄ haloalkyl, C₁₋₄ haloalkoxy, amino, C₁₋₄ alkylamino, C₂₋₈ dialkylamino, CN, NO₂, and OH;

each R^{A2} is independently selected from H, halo, C₁₋₄ alkyl, C₁₋₄ alkoxy, C₁₋₄ haloalkyl, C₁₋₄ haloalkoxy, amino, C₁₋₄ alkylamino, C₂₋₈ dialkylamino, CN, NO₂, and OH;

each R^{A2} is independently selected from H, C₁₋₄ alkyl, C₁₋₄ alkoxy, C₁₋₄ haloalkyl, C(O)R^{z′}, and C(O)OR^{z′}, wherein said C₁₋₄ alkyl is optionally substituted by phenyl, C₁₋₄ alkoxy, C₁₋₄haloalkoxy, CN, NO₂, or OH;

R^{z′} is H, C, alkyl, or phenyl;

each Cy¹ is independently selected from C₆₋₁₄ aryl, C₃₋₁₈ cycloalkyl, 5-16 membered heteroaryl, and 4-18 membered heterocycloalkyl, each of which is optionally substituted with 1, 2, 3, or 4 substituents independently selected from R^{Cy1};
each Cy² is independently selected from C₆₋₁₄ aryl, C₃₋₁₈ cycloalkyl, 5-16 membered heteroaryl, and 4-18 membered heterocycloalkyl, each of which is optionally substituted with 1, 2, 3, or 4 substituents independently selected from R^{Cy2};

each R^{Cy1} and R^{Cy2} is independently selected from halo, C₁₋₆ alkyl, C₁₋₄ haloalkyl, C₁₋₄ cyanoalkyl, C₂₋₆ alkenyl, C₂₋₆ alkynyl, phenyl, C₃₋₇ cycloalkyl, 5-6 membered heteroaryl, and 4-7 membered heterocycloalkyl, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}, wherein said C₁₋₆ alkyl, C₂₋₆ alkenyl, C₂₋₆ alkynyl, phenyl, C₃₋₇ cycloalkyl, 5-6 membered heteroaryl, and 4-7 membered heterocycloalkyl are each optionally substituted by 1, 2, 3, or 4 substituents independently selected from CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}SC(O)OR^{a5}, NR^{c5}C(O)NR^{c5}R^{d5}, NR^{c5}S(O)R^{b5}, NR^{c5}S(O)₂R^{b5}, NR^{e5}S(O)₂NR^{c5}R^{d5}, S(O)R^{b5}, S(O)NR^{c5}R^{d5}, S(O)₂R^{b5}, and S(O)₂NR^{c5}R^{d5};
each R^{a1}, R^{b1}, R^{c1}, R^{d1}, R^{a2}, R^{b2}, R^{c2}, R^{d2}, R^{a3}, R^{b3}, R^{c3}, R^{d3}, R^{a4}, R^{b4}, R^{c4}, R^{d4}, R^{a5}, R^{b5}, R^{c5}, and R^{d5} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl, C₃₋₁₀ cycloalkyl-C₁₋₆ alkyl, (5-10 membered heteroaryl)-C₁₋₆ alkyl, and (4-10 membered heterocycloalkyl)-C₁₋₆ alkyl, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl, C₃₋₁₀ cycloalkyl-C₁₋₆ alkyl, (5-10 membered heteroaryl)-C₁₋₆ alkyl, and (4-10 membered heterocycloalkyl)-C₁₋₆alkyl are each optionally substituted with 1, 2, 3, 4, or 5 substituents independently selected from R^{g};

each R^{e1}, R^{e2}, R^{e3}, R^{e4}, and R^{e5} is independently selected from H, C₁₋₄alkyl, and CN;

each R^{g} is independently selected from the group consisting of OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;

n is 0 or 1;

m is 0 or 1;

p is 0, 1, 2, or 3;

q is 0, 1, or 2;

a is 0 or 1; and

b is 0 or 1,

wherein any cycloalkyl or heterocycloalkyl group is optionally further substituted by 1 or 2 oxo groups.

In some embodiments, Y is O; Z is C(O)NR^{c3}R^{d3}; U is N; p is 0; and q is 0. In some embodiments W is N, and X is N. In some embodiments, A, B, D, and E are each independently selected from —CH₂— or —CH₂CH₂—.

In some embodiments, L is methylene or ethylene. In some embodiments, Cy is a linking phenyl, C₃₋₁₈ cycloalkyl, 5-10 membered heteroaryl, or 4-9 membered heterocycle.

Such compounds can be made according to methods known in the art, such as those described in U.S. Pat. No. 10,683,302, which is hereby incorporated by reference in its entirety.

In some embodiments, the MENIN inhibitor is SNDX-5613.

In some embodiments, the MENIN inhibitor is MI-1, MI-2, MI-2-2, MLS001171971-01, ML227, MCP-1, MI-503, MI-1481, MI-3454, MI-463, MI-136, MI-3, SNDX-5613, or VTP50469, or a pharmaceutically acceptable salt thereof.

Non-limiting examples of MENIN inhibitors are shown below:

| Name | Structure |
|---|---|
| MI-1 | 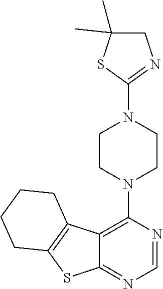 [SMILES: CC1(C)CN=C(N2CCN(c3ncnc4sc5c(c34)CCCC5)CC2)S1] |
| MI-2 | 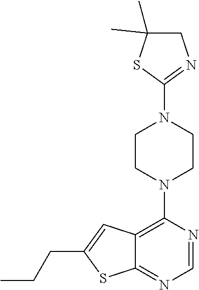 [SMILES: CCCc1cc2c(N3CCN(C4=NCC(C)(C)S4)CC3)ncnc2s1] |
| MI-2-2 | 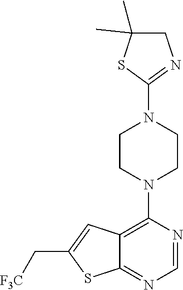 [SMILES: CC1(C)CN=C(N2CCN(c3ncnc4sc(CC(F)(F)F)cc34)CC2)S1] |
| MLS001171971-01 | 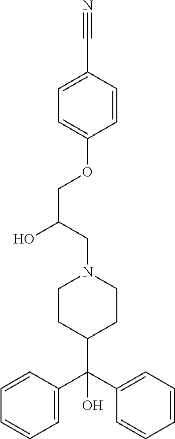 [SMILES: N#Cc1ccc(OCC(O)CN2CCC(C(O)(c3ccccc3)c3ccccc3)CC2)cc1] |
| ML227 | 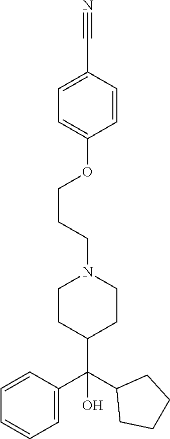 [SMILES: N#Cc1ccc(OCCCN2CCC(C(O)(c3ccccc3)C3CCCC3)CC2)cc1] |
| MCP-1 | 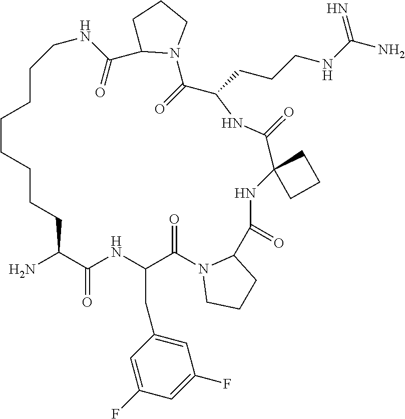 |
| MI-503 | 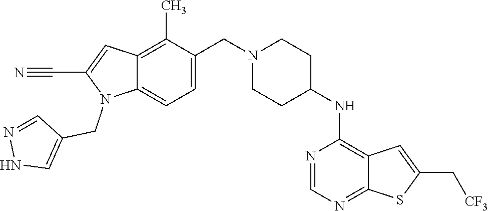 [SMILES: Cc1c(CN2CCC(Nc3ncnc4sc(CC(F)(F)F)cc34)CC2)ccc2c1cc(C#N)n2Cc1cn[nH]c1] |
| MI-1481 | 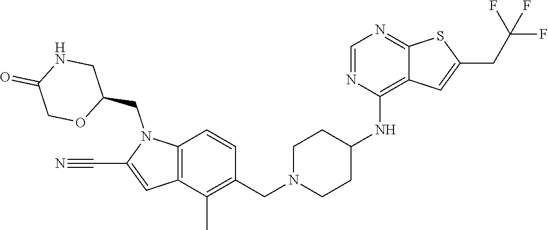 [SMILES: Cc1c(CN2CCC(Nc3ncnc4sc(CC(F)(F)F)cc34)CC2)ccc2c1cc(C#N)n2C[C@@H]1CNC(=O)CO1] |
| MI-3454 | 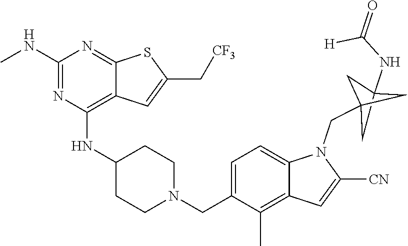 [SMILES: CNc1nc(NC2CCN(Cc3ccc4c(cc(C#N)n4CC45CC(NC=O)(C4)C5)c3C)CC2)c2cc(CC(F)(F)F)sc2n1] |
| MI-463 | 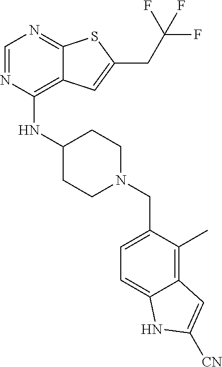 [SMILES: Cc1c(CN2CCC(Nc3ncnc4sc(CC(F)(F)F)cc34)CC2)ccc2[nH]c(C#N)cc12] |
| MI-136 | 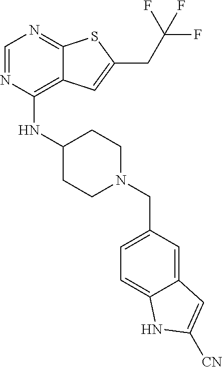 [SMILES: N#Cc1cc2cc(CN3CCC(Nc4ncnc5sc(CC(F)(F)F)cc45)CC3)ccc2[nH]1] |
| MI-3 | 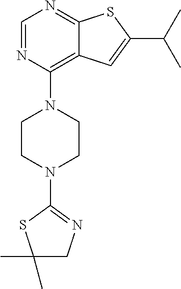 [SMILES: CC(C)c1cc2c(N3CCN(C4=NCC(C)(C)S4)CC3)ncnc2s1] |
| SNDX-5613 | 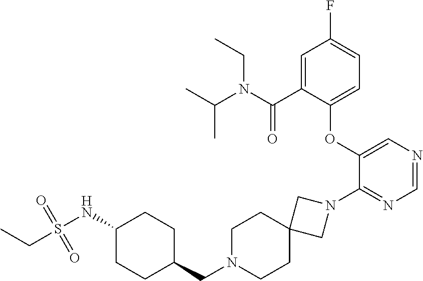 [SMILES: CCN(C(=O)c1cc(F)ccc1Oc1cncnc1N1CC2(CCN(C[C@H]3CC[C@H](NS(=O)(=O)CC)CC3)CC2)C1)C(C)C] |
| VTP50469 | 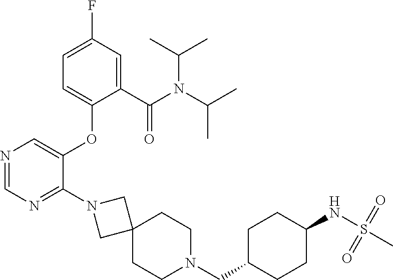 [SMILES: CC(C)N(C(=O)c1cc(F)ccc1Oc1cncnc1N1CC2(CCN(C[C@H]3CC[C@H](NS(C)(=O)=O)CC3)CC2)C1)C(C)C] |

Such compounds are commercially available or can be made according to methods known in the art.

In some embodiments, a MENIN inhibitor may be a gene editing system disrupting the MEN1 and/or KMT2A genomic sequence. In some embodiments, the MENIN inhibitor is one or more components of a gene editing system targeting one or more sites within a gene encoding MEN1 and/or KMT2A or a regulatory element thereof, a nucleic acid molecule encoding the one or more components of the gene editing system, or a combination thereof. In some embodiments, the BMI1 inhibitor is a gene editing system, and wherein the gene editing system is selected from the group consisting of CRISPR/Cas9, CRISPR/Cas13, a zinc finger nuclease system, a TALEN system, and a meganuclease system. In some embodiments, the gene editing system is a CRISPR/Cas9 system. In some embodiments, the gene editing system is a CRISPR/Cas13 system. In some embodiments, the gene editing system is a zinc finger nuclease system. In some embodiments, the gene editing system is a TALEN system. In some embodiments, the gene editing system is a meganuclease system. In some embodiments, the MENIN inhibitor comprises a guide RNA molecule comprising a tracr and a crRNA. In some embodiments, the crRNA comprises a targeting domain that is complementary with a target sequence of MEN1 and/or KMT2A.

Also provided herein, in some embodiments, are pharmaceutical compositions that comprise compounds as described herein, or pharmaceutically acceptable salts thereof, and one or more pharmaceutically acceptable vehicles selected from carriers, adjuvants, and excipients.

Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses. The pharmaceutical composition may be administered by various methods including, for example, rectal, buccal, intranasal, and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein. Administration may be via, for example, capsule or enteric coated tablets. In making the pharmaceutical compositions that include at least one compound described herein, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound described herein can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

Kits

Provided herein are kits that include a compound of the disclosure and suitable packaging. In one embodiment, a kit further includes instructions for use. In one aspect, a kit includes a compound of the disclosure and a label and/or instructions for use of the compounds in the treatment of the indications, including the diseases or conditions, described herein.

Provided herein are also articles of manufacture that include a compound described herein in a suitable container. The container may be a vial, jar, ampoule, preloaded syringe, and intravenous bag.

Dosing

The specific dose level of a compound of the present disclosure for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The daily dosage may also be described as a total amount of a compound described herein administered per dose or per day. Daily dosage of a compound described herein may be between about 1 mg and 4,000 mg, between about 2,000 to 4,000 mg/day, between about 1 to 2,000 mg/day, between about 1 to 1,000 mg/day, between about 10 to 500 mg/day, between about 20 to 500 mg/day, between about 50 to 300 mg/day, between about 75 to 200 mg/day, or between about 15 to 150 mg/day.

When administered orally, the total daily dosage for a human subject may be between 1 mg and 1,000 mg, between about 1,000-2,000 mg/day, between about 10-500 mg/day, between about 50-300 mg/day, between about 75-200 mg/day, or between about 100-150 mg/day.

The compounds of the present disclosure or the compositions thereof may be administered once, twice, three, or four times daily, using any suitable mode described above. Also, administration or treatment with the compounds may be continued for a number of days; for example, commonly treatment would continue for at least 7 days, 14 days, or 28 days, for one cycle of treatment. Treatment cycles are well known, and are frequently alternated with resting periods of about 1 to 28 days, commonly about 7 days or about 14 days, between cycles. The treatment cycles, in other embodiments, may also be continuous.

It is understood that modifications which do not substantially affect the activity of the various embodiments of this disclosure are also included within the definition of the disclosure provided herein. Accordingly, the following examples are intended to illustrate but not limit the present disclosure.

### EXAMPLES

### Methods

Knockdown Studies

For knocking down Bmi1, pLKO.1-blast lentiviral shRNA constructs containing Bmi1-specific targeting sequences (Bmi1-sh1, SEQ ID 1: GCA GAT TGG ATC GGA AAG TAA; Bmi1-sh2, SEQ ID 2: GAT GAG AAG AGG ATT ATA) and another non-targeting control sequence (NC-sh, SEQ ID 3: CCT AAG GTT AAG TCG CCC TCG) were used. For Men1 knockdown studies, pLKO.5-puro lentiviral shRNA constructs carrying Men1-specific targeting sequences (Men1-sh1, SEQ ID 4: CGA TCT TCA CAC TGA CTC TTT; Men1-sh2, SEQ ID 5: TAC CAC TGT CGC AAC CGA AAT) and a non-targeting control sequence (NC-sh, SEQ ID 6: CAA GAT GAA GAG CAC CAA) were used. For Kmt2a knockdown studies, pLKO.1-puro lentiviral shRNA constructs carrying Kmt2a-specific targeting sequences (Kmt2a-sh1, SEQ ID 7: CGC CTT CAC TTG ACC ATA ATT; Kmt2a-sh2, SEQ ID 8: GCA CTT GAA GAC TTC TAA) and the same non-targeting control sequence as above were used.

To generate infectious lentivirus, the constructs were co-transfected using LipoD293 transfection reagent into HEK293T cells along with packaging plasmids psPAX2 and pMD2.G, and virus were harvested at 72 hours after transfection. Lentiviral infections were performed by spinoculation in which a mixture of lentivirus and MEL cells in 24-well plates were spun at 2000×g for 90 minutes at 37° C. Puromycin (3 μg/ml) or blasticidin (40 μg/ml) was added to culture medium (RPMI 1640 medium with 10% fetal bovine serum) at 24 hours after spinoculation to select for infected cells. Infected MEL cells were harvested at 96 hours after infection for analyzing relative mRNA levels of Hbb-y and Hbb-bh1 using real-time RT-PCR.

MEL Cells

For studies testing inhibitors on MEL cells, the cells were treated with selected inhibitors or vehicle in culture medium (RPMI 1640 medium with 10% fetal bovine serum) for 48 hours and were subsequently harvested and analyzed for relative mRNA levels of Hbb-bh1 and Hbb-y using real-time RT-PCR.

Primers as used in the Examples as described herein are summarized below.

| Primers | Sequence | Species |
|---|---|---|
| Hbb-y Forward; | 5′-TGGCCTGTGGAGTAAGGT | Mouse |
| SEQ ID 9 | CAA-3′ | |
| Hbb-y Reverse; | 5′-GAAGCAGAGGACAAGTTC | Mouse |
| SEQ ID 10 | CCA-3′ | |
| Hbb-bh1 Forward; | 5′-GAAACCCCCGGATTAGAG | Mouse |
| SEQ ID 11 | CC-3′ | |
| Hbb-bh1 Reverse; | 5′-GAGCAAAGGTCTCCTTGA | Mouse |
| SEQ ID 12 | GGT-3′ | |
| Hbb-b1/2 Forward; | 5′-GCACCTGACTGATGCTGA | Mouse |
| SEQ ID 13 | GAA-3′ | |
| Hbb-b1/2 Reverse; | 5′-TTCATCGGCGTTCACCTT | Mouse |
| SEQ ID 14 | TCC-3′ | |
| Rpl4 Forward; | 5′-ATGATGAACACCGACCTT | Mouse |
| SEQ ID 15 | AGCA-3′ | |
| Rpl4 Reverse; | 5′-CGGAGGGCTCTTTGGATT | Mouse |
| SEQ ID 16 | TC-3′ | |
| g-globin Forward; | 5′-TGGATGATCTCAAGGGCA | Human |
| SEQ ID 17 | C-3′ | |
| g-globin Reverse; | 5′-TCAGTGGTATCTGGAGGA | Human |
| SEQ ID 18 | CA-3′ | |
| e-globin Forward; | 5′-GCAAGAAGGTGCTGACTT | Human |
| SEQ ID 19 | CC-3′ | |
| e-globin Reverse; | 5′-ACCATCACGTTACCCAGG | Human |
| SEQ ID 20 | AG-3′ | |
| b-globin Forward; | 5′-CTGAGGAGAAGTCTGCCG | Human |
| SEQ ID 21 | TTA-3′ | |
| b-globin Reverse; | 5′-AGCATCAGGAGTGGACAG | Human |
| SEQ ID 22 | AT-3′ | |
| GAPDH Forward; | 5′-ACCCAGAAGACTGTGGAT | Human |
| SEQ ID 23 | GG-3′ | |
| GAPDH Reverse; | 5′-TTCAGCTCAGGGATGACC | Human |
| SEQ ID 24 | TT-3′ | |

Erythroid Differentiation and Treatment of Human CD34⁺ HSPCs

Primary human mobilized CD34⁺ HSPCs (StemCell Technologies Inc.) were initially cultured in expansion medium containing StemSpan-XF Medium (StemCell Technologies Inc.) with 1× CC 100 cytokine mix (StemCell Technologies Inc.). Cells were maintained in this expansion medium at a density of 0.1-1×10⁶ cells/ml for a total of 4 days with media changes every two days. On day 4, cells were induced to undergo erythroid differentiation in three phases as known in the art. Briefly, base medium for differentiation contains Iscove's Modified Dulbecco's Medium plus 2% human peripheral blood plasma (Innovative Research), 3% human AB serum (Sigma), 200 mg/mL human holo-transferrin (Sigma), 3 IU/mL heparin (Sigma), 10 mg/mL human insulin (Sigma), and 1% Penicillin-Streptomycin (Life Technologies Inc.). In the first phase (day 0 to day 6), cells were cultured at a concentration of 10⁵ cells/ml in the presence of 10 ng/mL human stem cell factor (Biolegend), 1 ng/mL human IL-3 (Biolegend), and 3 IU/mL human erythropoietin (Janssen). In the second phase (day 7 to day 11), IL-3 was omitted from the first phase culture medium. In the third phase (day 12 to day 16), both stem cell factor and IL-3 were omitted and the concentration of transferrin was adjusted to 1 mg/ml. Cell density also was adjusted to 10⁶ cells/ml in the beginning of this phase. Drug treatments were started at day 2 of expansion condition and maintained thereafter. New drugs were added every 1-2 days with medium change.

### Example 1: BMI1

To test whether BMI1 may play a role in the repression of embryonic and fetal hemoglobin expression, the expression levels of Bmi1 were knocked down using two different lentiviral shRNAs in MEL cells, which is a mouse erythroid progenitor cell line commonly used for studying repression of embryonic and fetal hemoglobins.

Bmi1 knockdowns significantly increased the mRNA levels of mouse embryonic globin genes Hbb-y and Hbb-bh1 by up to nearly 60-fold and 20-fold respectively (FIG. 1). These results identify BMI1 as a critical repressor of embryonic globin gene transcription and also a novel therapeutic target for inducing embryonic and fetal hemoglobin expression.

PTC596, a known BMI1 inhibitor, was evaluated for its ability to induce the expression of Hbb-bh1 and Hbb-y in MEL cells.

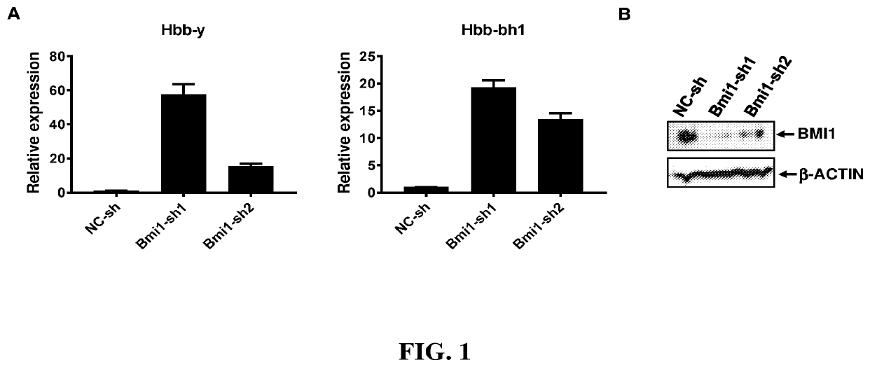

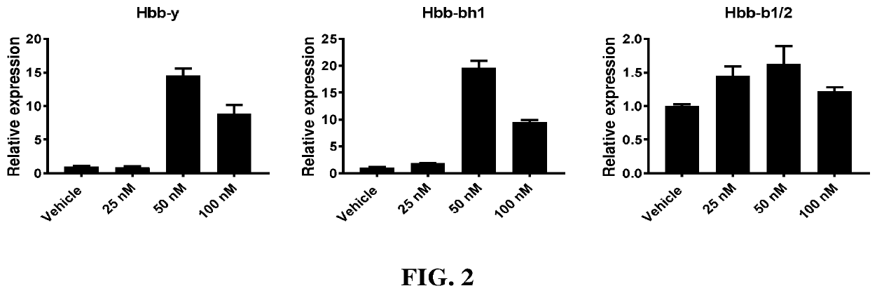

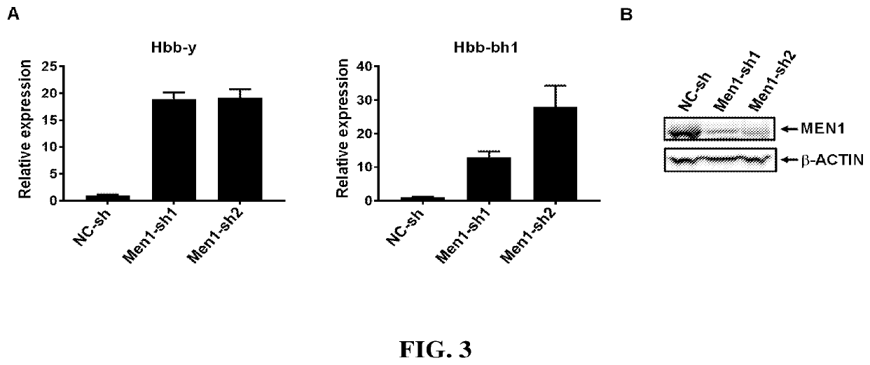

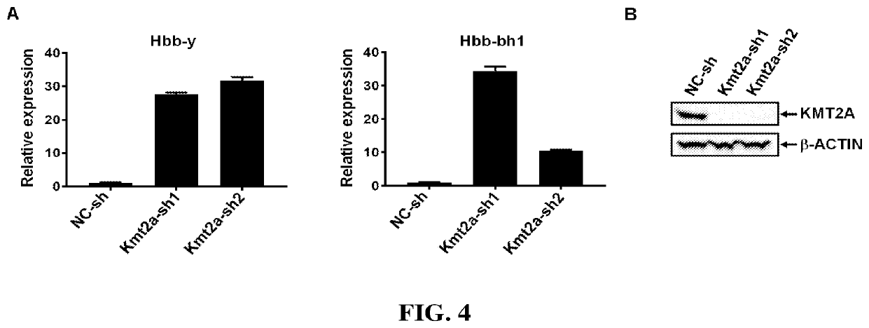

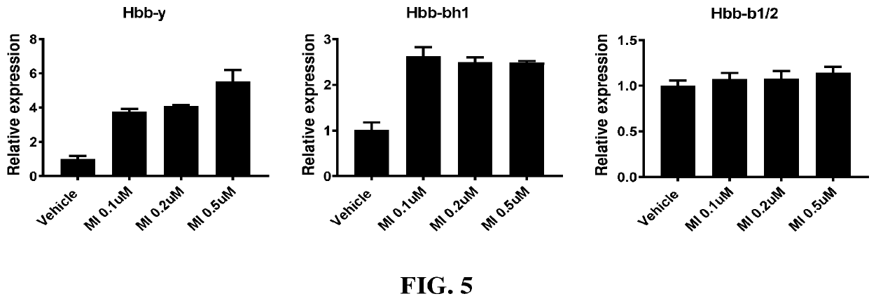

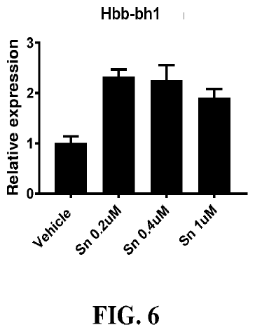

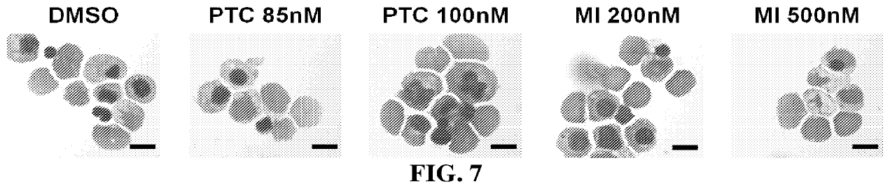

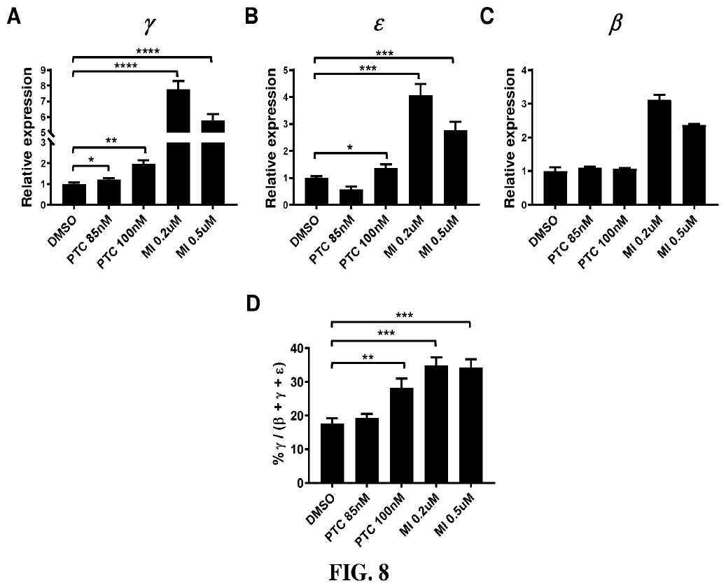

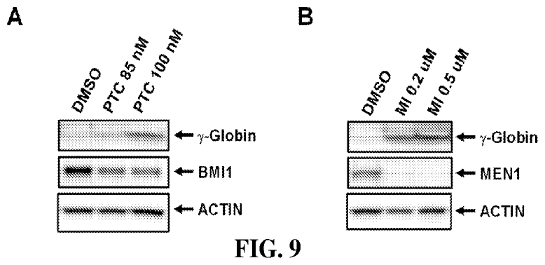

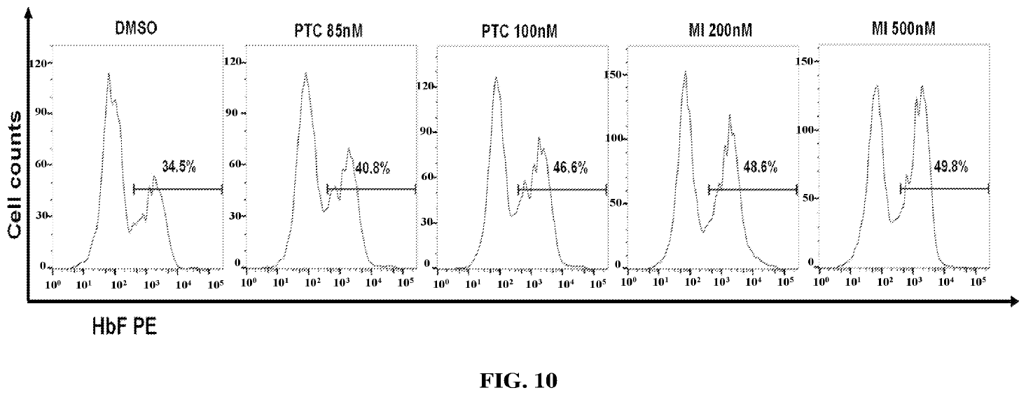

Consistent with the knockdown study, treatment with 50 nM of PTC596 for 48 hours increased mRNA levels of Hbb-y and Hbb-bh1 by nearly 15- and 20-fold respectively (FIG. 2). In contrast, PTC596 exerted minimal effects on the expression levels of Hbb-b1/2 (adult hemoglobin) (FIG. 2). These results suggest that PTC596 is efficient in activating embryonic globin expression in erythroid cells.

### Example 2: MEN1 and KMT2A

To test whether MEN1 may play a role in the repression of embryonic and fetal hemoglobin expression, the expression levels of Men1 using two different lentiviral shRNAs in MEL cells were knocked down, which are a mouse erythroid progenitor cell line commonly used for studying repression of embryonic and fetal hemoglobins.

Men1 knockdowns significantly increased the mRNA levels of embryonic globin genes Hbb-y and Hbb-bh1 by up to nearly 20- and 30-fold, respectively (FIG. 3). These results identify MEN1 as a critical repressor of embryonic and fetal globin gene transcription and also a novel therapeutic target for inducing embryonic and fetal hemoglobin expression.

Since MEN1 is known to regulate transcription through physical interaction with KMT2A, whether KMT2A is also involved in the repression of embryonic and fetal globin expression was also tested by knocking down Kmt2a in MEL cells.

Kmt2a knockdowns also significantly increased the mRNA levels of both Hbb-y and Hbb-bh1 by up to over 30-fold (FIG. 4), indicating that KMT2A also represents a novel critical repressor of embryonic and fetal globin gene transcription and could be targeted to induce embryonic and fetal hemoglobin expression.

MI-3454 and SNDX-5613, known to block the interaction of MEN1 with KMT2A, were evaluated for their ability to induce the expression of Hbb-bh1 and Hbb-y.

Consistent with the knockdown studies, treatment with MI-3454 at tested concentrations for 48 hours led to significant increases in the mRNA levels of both Hbb-bh1 and Hbb-y in MEL cells (FIG. 5). The activating effect of MI-3454 appears limited to mouse embryonic globins as only minor changes on the levels of adult globin, Hbb-b1/2, were detected with the same treatments (FIG. 5). Treatments with SNDX-5613 also significantly increased the expression of Hbb-bh1 in MEL cells (FIG. 6).

These results further suggest that MENIN inhibitors, such as MI-3454 and SNDX-5613, could be used for treating patients of SCD and β-thalassemia due to their activity in activating fetal and/or embryonic hemoglobin expression.

### Example 3: Effects of PTC596 and MI-3454 on Human Fetal and Embryonic Hemoglobin Expression

The effects of PTC596 and MI-3454 on human fetal (γ) and embryonic (ε) globin expression in erythroid cells produced in culture from primary human CD34⁺ hematopoietic stem and progenitor cells (HSPCs) were further tested. Human CD34⁺ HSPCs mobilized by granulocyte-colony stimulating factor (G-CSF) were first expanded in culture for 4 days and subsequently induced to undergo erythroid differentiation for 14 days in conditions as known in the art. PTC596, MI-3454, or control DMSO were added to the culture from day 2 of the expansion phase and were replenished upon media change every two days. Similar to the DMSO-treated control culture, many maturing erythroblasts and enucleated reticulocytes were observed in the inhibitors-treated cultures after 14 days of differentiation (FIG. 7), suggesting that erythroid differentiation was not blocked by either inhibitor.

Treatment with PTC-596 or MI-3454 significantly increased γ- and α-globin mRNA levels in these cells (FIG. 8A and FIG. 8B). Although MI-3454 also induced an increase in β-globin mRNA levels (FIG. 8C), both inhibitors were able to induce a significant increase in the percentage of γ-globin expression levels in total β-like globin (β+γ+ε) expression levels (FIG. 8D).

In agreement with the increases in γ-globin mRNA levels, increased μ-Globin protein levels were detected in cells treated with the inhibitors (FIG. 9). Consistent with their target specificities, PTC596 and MI-3454 also reduced BMI1 and MENIN protein levels in these cells respectively (FIG. 9). In addition, cultures treated with these inhibitors also contain higher percentage of F-cells (FIG. 10), suggesting the treatments also induced more cells to express significant amount of HbF.

It is contemplated that MEN1, KMT2A, and BMI1 are therapeutic targets for SCD and β-thalassemia. It is further contemplated that the results described herein show that BMI1 inhibitors, such as PTC596, and MENIN inhibitors, such as MI-3454, may be used for treating patients of SCD and β-thalassemia due to their activity in activating fetal and embryonic hemoglobin expression.

Although the invention has been described with reference to the disclosed embodiments, those skilled in the art will readily appreciate that the specific examples and studies detailed above are only illustrative of the invention. It should be understood that various modifications can be made without departing from the spirit of the invention. Accordingly, the invention is limited only by the following claims.

What is claimed is:

1. A method for treating sickle cell disease in a subject in need thereof comprising administering to the subject a therapeutically effective amount of a compound that modulates activity of BMI1 protein.

2. A method for treating sickle cell disease in a subject in need thereof comprising administering to the subject a pharmaceutical composition comprising a therapeutically effective amount of a compound that modulates activity of BMI1 protein and a pharmaceutically acceptable carrier.

3. A method for treating β-thalassemia in a subject in need thereof comprising administering to the subject a therapeutically effective amount of a compound that modulates activity of BMI1 protein.

4. A method for treating β-thalassemia in a subject in need thereof comprising administering to the subject a pharmaceutical composition comprising a therapeutically effective amount of a compound that modulates activity of BMI1 protein and a pharmaceutically acceptable carrier.

5. The method of any one of the preceding claims, wherein a compound that modulates activity of BMI1 protein is a compound that degrades the BMI1 protein.

6. A method for treating sickle cell disease in a subject in need thereof comprising administering to the subject a therapeutically effective amount of a compound that inhibits activity of BMI1 protein.

7. A method for treating sickle cell disease in a subject in need thereof comprising administering to the subject a pharmaceutical composition comprising a therapeutically effective amount of a compound that inhibits activity of BMI1 protein and a pharmaceutically acceptable carrier.

8. A method for treating β-thalassemia in a subject in need thereof comprising administering to the subject a therapeutically effective amount of a compound that inhibits activity of BMI1 protein.

9. A method for treating β-thalassemia in a subject in need thereof comprising administering to the subject a pharmaceutical composition comprising a therapeutically effective amount of a compound that inhibits activity of BMI1 protein and a pharmaceutically acceptable carrier.

10. The method of any one of claims 6-9, wherein the compound that inhibits activity of BMI1 protein is PTC596.

11. A method for treating sickle cell disease in a subject in need thereof comprising administering to the subject a therapeutically effective amount of a compound that modulates the activity of Mixed Lineage Leukemia (MLL) complex.

12. A method for treating sickle cell disease in a subject in need thereof comprising administering to the subject a pharmaceutical composition comprising a therapeutically effective amount of a compound that modulates the activity of MLL complex and a pharmaceutically acceptable carrier.

13. A method for treating β-thalassemia in a subject in need thereof comprising administering to the subject a therapeutically effective amount of a compound that modulates the activity of MLL complex.

14. A method for treating β-thalassemia in a subject in need thereof comprising administering to the subject a pharmaceutical composition comprising a therapeutically effective amount of a compound that modulates the activity of MLL complex and a pharmaceutically acceptable carrier.

15. The method of any one of claims 11-14, wherein a compound that modulates the activity of Mixed Lineage Leukemia (MLL) complex is a compound that inhibits the MLL complex.

16. A method for treating sickle cell disease in a subject in need thereof comprising administering to the subject a therapeutically effective amount of a compound that inhibits activity of MLL complex.

17. A method for treating sickle cell disease in a subject in need thereof comprising administering to the subject a pharmaceutical composition comprising a therapeutically effective amount of a compound that inhibits activity of MLL complex and a pharmaceutically acceptable carrier.

18. A method for treating β-thalassemia in a subject in need thereof comprising administering to the subject a therapeutically effective amount of a compound that inhibits activity of MLL complex.

19. A method for treating β-thalassemia in a subject in need thereof comprising administering to the subject a pharmaceutical composition comprising a therapeutically effective amount of a compound that inhibits activity of MLL complex and a pharmaceutically acceptable carrier.

20. The method of any one of claims 15-19, wherein a compound that inhibits the activity of MLL complex is a compound that inhibits activity of MEN1 protein.

21. The method of any one of claims 15-19, wherein a compound that inhibits the activity of MLL complex is a compound that inhibits activity of KMT2A protein.

22. The method of any one of claims 15-19, wherein a compound that inhibits the MLL complex is a compound that inhibits interaction between MEN1 protein and KMT2A protein.

22. The method of claim 22, wherein the compound that inhibits interaction between MEN1 protein and KMT2A protein is MI-3454.

23. The method of any one of the preceding claims, further comprising increasing embryonic hemoglobin expression.

24. The method of any one of the preceding claims, further comprising increasing fetal hemoglobin expression.
